SEQUENCE LISTING (30 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - DNA, 8899 bp, Artificial
  Factor VIII-500 in plasmid pTT5
gtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgaattccctctagagggcccgtttctgctagcaagcttgctagcggccgcgtttaactggtaagtttagtctttttgtcttttatttcaggtcccggatcgaagcttggctgcaggtcgacgccaccatggaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcgcgacaccctagccaaaacccaccggtcttgaaacgccatcaacgggagatcactcgtactactcttcagtctgatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgaggatccccaaacgctagagcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgaaccctaaacgggtagcatatgcttcccgggtagtagtatatactatccagactaaccctaattcaatagcatatgttacccaacgggaagcatatgctatcgaattagggttagtaaaagggtcctaaggaacagcgatgtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 3 - DNA, 34 bp, Artificial
  Synthetic primer
cgctcagttgccaagtgtcatcctaaaacttggg

SEQ ID NO: 4 - DNA, 34 bp, Artificial
  Synthetic primer
cccaagttttaggatgacacttggcaactgagcg

SEQ ID NO: 5 - DNA, 32 bp, Artificial
  Synthetic primer
tttatggcatacacaaatgaaacctttaagac

SEQ ID NO: 6 - DNA, 32 bp, Artificial
  Synthetic primer
gtcttaaaggtttcatttgtgtatgccataaa

SEQ ID NO: 7 - DNA, 41 bp, Artificial
  Synthetic primer
ggcatacacagatgaatgctttaagactcgtgaagctattc

SEQ ID NO: 8 - DNA, 41 bp, Artificial
  Synthetic primer
gaatagcttcacgagtcttaaagcattcatctgtgtatgcc

SEQ ID NO: 9 - DNA, 43 bp, Artificial
  Synthetic primer
ggcatacacagatgaaaactttacgactcgtgaagctattcag

SEQ ID NO: 10 - DNA, 43 bp, Artificial
  Synthetic primer
ctgaatagcttcacgagtcgtaaagttttcatctgtgtatgcc

SEQ ID NO: 11 - DNA, 37 bp, Artificial
  Synthetic primer
gatgtccgtcctaattattcaaggagattaccaaaag

SEQ ID NO: 12 - DNA, 37 bp, Artificial
  Synthetic primer
cttttggtaatctccttgaataattaggacggacatc

SEQ ID NO: 13 - DNA, 40 bp, Artificial
  Synthetic primer
gatgtccgtcctttgtattgtaggagattaccaaaaggtg

SEQ ID NO: 14 - DNA, 40 bp, Artificial
  Synthetic primer
caccttttggtaatctcctacaatacaaaggacggacatc

SEQ ID NO: 15 - DNA, 37 bp, Artificial
  Synthetic primer
gtccgtcctttgtataacaggacattaccaaaaggtg

SEQ ID NO: 16 - DNA, 37 bp, Artificial
  Synthetic primer
caccttttggtaatgtcctgttatacaaaggacggac

SEQ ID NO: 17 - DNA, 42 bp, Artificial
  Synthetic primer
gattaccaaaaggtgtatgccatttgaaggattttccaattc

SEQ ID NO: 18 - DNA, 42 bp, Artificial
  Synthetic primer
gaattggaaaatccttcaaatggcatacaccttttggtaatc

SEQ ID NO: 19 - DNA, 40 bp, Artificial
  Synthetic primer
ccaaaaggtgtaaatcattcgaaggattttccaattctgc

SEQ ID NO: 20 - DNA, 40 bp, Artificial
  Synthetic primer
gcagaattggaaaatccttcgaatgatttacaccttttgg

SEQ ID NO: 21 - DNA, 45 bp, Artificial
  Synthetic primer
gaaggattttccaatttgcccaggagaaatattcaaatataaatg

SEQ ID NO: 22 - DNA, 45 bp, Artificial
  Synthetic primer
catttatatttgaatatttctcctgggcaaattggaaaatccttc

SEQ ID NO: 23 - DNA, 40 bp, Artificial
  Synthetic primer
gccgccggcatatgaagctatacatttacgatcactgccc

SEQ ID NO: 24 - DNA, 46 bp, Artificial
  Synthetic primer
ccgccgccctcgagaatcgccattgatgataacaaattgatttgtg

SEQ ID NO: 25 - DNA, 88 bp, Artificial
  Synthetic primer
aattcgccaccatgccctcgagcgtctcgtggggcatcctcctgctggcaggcctgtgctgcctggtccctgtctcgctagctctgca

SEQ ID NO: 26 - DNA, 80 bp, Artificial
  Synthetic primer
gagctagcgagacagggaccaggcagcacaggcctgccagcaggaggatgccccacgagacgctcgagggcatggtggcg

SEQ ID NO: 27 - DNA, 33 bp, Artificial
  Synthetic primer
cgctagctaaaacttgggtacattacattgctg

SEQ ID NO: 28 - DNA, 36 bp, Artificial
  Synthetic primer
agcggccgctctaacaactagaaaccttcagtaagg

SEQ ID NO: 29 - DNA, 24 bp, Artificial
  Synthetic primer
ggagtgcagcttgaggatccagag

SEQ ID NO: 30 - DNA, 75 bp, Artificial
  Synthetic primer
agcggccgctctatttgccatcaatcaggcgcggatccacctgatcttcgccggagaagcttcttggttcaatgg

(19) United States
(12) Patent Application Publication
(10) Pub. No.: US 2008/0227691 A1
(43) Pub. Date: Sep. 18, 2008

(54) Blood Coagulation FVIII Analogues

(75) Inventors: Henrik Ostergaard, Olstykke (DK); Gert Bolt, Vaerlose (DK); Thomas Dock Steenstrup, Gentofte (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Health Care AG, Zurich (CH)

(21) Appl. No.: 11/910,349

(22) PCT Filed: Apr. 3, 2006
(86) PCT No.: PCT/EP06/61275

§ 371 Date: May 2, 2008

(30) Foreign Application Priority Data
Apr. 1, 2005 (DK) PA 2005 00462

(51) Int. Cl. A61K 38/37; C07K 14/755; A61P 7/00

(52) U.S. Cl. 514/8; 530/383

(57) ABSTRACT

The invention is related to a FVIII analogue which has a circulation time in the blood stream before activation of at least about two times of that of native FVIII and a week after injection to a patient retains at least about 5% of the FVIII activity compared to the initial activity peak value reached after injection. The claimed FVIII analogues comprise a targeted disruption of one or more of the clearance sites in the FVIII molecule by introduction of at least one N-glycosylation site or by introduction of at least one Cys residue within or spatially close to the clearance site in the A2 domain or a combination thereof. The inserted cysteine residues may be further modified by conjugation with a chemical group increasing the molecular weight of the FVIII analogue.

### FIELD OF THE INVENTION

The present invention is related to certain blood coagulation FVIII analogues and derivatives with a prolonged circulation time in the blood stream compared to native FVIII.

### BACKGROUND OF THE INVENTION

Haemophilia A is an inherited bleeding disorder caused by deficiency or dysfunction of coagulation factor VIII (FVIII) activity. The disease is treated by intravenously injection of coagulation factor FVIII which is either isolated from blood or produced recombinantly.

FVIII is an essential component of the intrinsic coagulation pathway. Activated FVIII (FVIIIa) is a co-factor for activated FIX (FIXa), which converts factor X (FX) to activated FX (FXa). FXa in turn converts prothrombin to thrombin—the crucial factor in clot formation. The effect of the FVIIIa/FIXa complex is therefore to amplify the thrombin generation that has already been initiated by the extrinsic pathway.

FVIII is a large, complex glycoprotein that primarily is produced by hepatocytes. FVIII consists of 2351 amino acids, including signal peptide, and contains several distinct domains, as defined by homology. There are three A-domains, a unique B-domain, and two C-domains. The domain order can be listed as NH₂-A1-A2-B-A3-C1-C2-COOH. FVIII circulates in plasma as two chains, separated at the B-A3 border. The chains are connected by bivalent metal ion-bindings. The A1-A2-B chain is termed the heavy chain (HC) while the A3-C1-C2 is termed the light chain (LC). The B-domain is cleaved at several different sites, generating large heterogeneity in plasma FVIII. The exact function of the heavily glycosylated B-domain is unknown and the domain is dispensable for FVIII activity.

FVIII is secreted as a 2332 amino acid protein with the domain architecture A1-A2-B-A3-C1-C2. Subsequent processing generates the active heterotrimer composed of 50 (A1), 43 (A2), and 73 kDa (A3-C1-C2) fragments. Although the nature of the interactions maintaining the three subunits together have not been completely established, it is currently believed that a metal ion links the A1 and A3-C1-C2 subunits, while A2 is likely to interact primarily with the A1 subunit.

The circulatory half life of FVIII is 12-14 hours. Although complexation with vWf is crucial for maintenance of normal levels of FVIII in circulation, clearance appears to be mediated by several other pathways involving recognition of LRP (low density lipoprotein receptor-related protein) and HSPG (heparan sulphate proteoglycan) binding-sites on the molecule. FVIII contains at least two LRP recognition sites, one in the A2 domain comprising residues 484-509 and one in A3 (residues 1811-1818), whereas a single HSPG site has been localized to residues 558-565 in A2.

Evidence in favour of a role of these recognition sites in the clearance of FVIII has come from the observation that antagonizing LRP-dependent degradation in mice prolonged the circulatory half-life of FVIII by 3.5-fold. Additional blocking of HSPG-facilitated degradation led to a further 1.6-fold increase in half-life.

Haemophilia A can be caused by mutations, re-arrangements, or deletions in the FVIII gene, leading to FVIII protein deficiency or secretion of functionally defect FVIII protein. The clinical manifestation is not on primary haemostasis—formation of the blood clot occurs normally—but the clot is unstable due to a lack of secondary thrombin formation.

Current treatment recommendations are moving from traditional on-demand treatment towards prophylaxis. Prophylaxis, which enables a virtually symptom-free life for the patients, puts dosing requirements at several doses a week.

It has been suggested to make a protracted FVIII derivative in WO 03/31464. By uniquely linking PEG polymers to sugar chains, at sites that are removed from the peptide chain, GlycoPEGylation helps preserve the bioactivity of the protein while still extending its half-life.

WO 00/71714 and WO 02/060951 disclose certain mutant FVIII in the A2 domain with increased half-life. U.S. Pat. No. 6,759,216 discloses FVIII mutants wherein FVIII is glycosylated at sites that are known to be antibody recognition epitopes by replacing the Leu in position 486 in the A2 domain with Asn. U.S. Pat. No. 5,859,204 describes mutants of human factor VIII having reduced antigenicity and reduced immunoreactivity.

### SUMMARY OF THE INVENTION

In one aspect the present invention is related to a FVIII analogue having a circulation time in the blood stream before activation of at least about two times of that of human FVIII.

In another aspect the present invention is related to a FVIII analogue which a week after injection to a patient retains at least about 5% of the FVIII activity compared to the initial activity peak value reached after injection.

In one embodiment the present invention relates to targeted disruption of one or more of the clearance sites in the FVIII molecule by introduction of at least one N-glycosylation site and/or by introduction of at least one Cys residue within or spatially close to the clearance site in the A1 and or A2 domain of human factor FVIII.

In an additional aspect the invention is related to subsequent chemical modification of the introduced cysteine residue(s) within or spatially close to the clearance site. Thus one or more of the amino acid residues in the A1 and/or A2 domain of the human FVIII molecule may be substituted with another amino acid residue. The inserted amino acid residue(s) may then be further derivatized by attachment of bulky chemical groups which will interfere with the clearance of the FVIII molecule.

In one embodiment the present invention is related to a factor VIII analogue comprising an amino acid substitution of at least one of the natural amino acid residues in positions 20-29, 268-276, 302-313, 321-326, 333-395, 430-520, 528-554, 559-564, 571-593 and/or 638-643 of the native FVIII molecule, which amino acid substitution(s) result(s) in a factor VIII analogue with a LRP binding affinity lower than that of human factor VIII and factor VIII activity being substantially the same as the activity of activated human factor VIII.

In one embodiment the present invention is related to a factor VIII analogue comprising one or more amino acid substitutions in residues 20-29 of the native FVIII molecule In one embodiment the present invention is related to a factor VIII analogue comprising one or more amino acid substitutions in residues 268-276 of the native FVIII molecule In one embodiment the present invention is related to a factor VIII analogue comprising one or more amino acid substitutions in residues 302-313 of the native FVIII molecule.

In one embodiment the present invention is related to a factor VIII analogue comprising one or more amino acid substitutions in residues 321-326 of the native FVIII molecule.

In one embodiment the present invention is related to a factor VIII analogue comprising one or more amino acid substitutions in residues 333-395 of the native FVIII molecule.

In one embodiment the present invention is related to a factor VIII analogue comprising one or more amino acid substitutions in residues 420-520 of the native FVIII molecule.

In one embodiment the present invention is related to a factor VIII analogue comprising one or more amino acid substitutions in residues 528-554 of the native FVIII molecule.

In one embodiment the present invention is related to a factor VIII analogue comprising one or more amino acid substitutions in residues 559-564 of the native FVIII molecule.

In one embodiment the present invention is related to a factor VIII analogue comprising one or more amino acid substitutions in residues 571-593 of the native FVIII molecule.

In one embodiment the present invention is related to a factor VIII analogue comprising one or more amino acid substitutions in residues 638-643 of the native FVIII.

In another embodiment the present invention is related to a factor VIII analogue comprising one or more amino acid substitutions in residues 20-29, 268-276, 302-313, 321-326, 333-395, 430-520, 528-554, 559-564, 571-593 and/or 638-643 of the native FVIII molecule which amino acid substitutions result in one or more N-glycan consensus site(s) and/or one or more cysteine residue(s).

In a further embodiment the present invention is related to a factor VIII analogue wherein the inserted cysteine amino acid(s) residue substitutions are conjugated with a chemical group increasing the molecular weight of the Factor VIII analogue.

In one embodiment the present invention is related to a factor VIII analogue comprising one or more amino acid substitutions in residues 20-29 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 268-276 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 302-313 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 321-326 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 333-395 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 430-520 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 528-554 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 559-564 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 571-593 of the native FVIII molecule which amino acid substitutions result in one or more N-glycan consensus site(s) and/or one or more cysteine residue(s).

In one embodiment the present invention is related to a factor VIII analogue comprising one or more amino acid substitutions in residues 638-643 of the native FVIII molecule which amino acid substitutions result in one or more N-glycan consensus site(s) and/or one or more cysteine residue(s).

In another embodiment at least one inserted cysteine amino acid residue is conjugated with a chemical group increasing the molecular weight of the Factor VIII polypeptide.

In one embodiment the amino acid residue substitutions result in one or more N-glycan consensus sites.

In another embodiment the amino acid residue substitution is a substitution of one or more of the natural amino acid residues with a cystein residue.

In a further embodiment the invention is related to a factor VIII analogue wherein at least one of the natural amino residues in position

i) L24, D27, F276, F306, L307, Y323, W382, H384, Y385, E389, W393, R427, M429, A430, I442, E445, I448, E456, V457, A469, Y476, T481, D482, R484, K499, T514, E518, V537, N538, A544, G546, I548, N572, F576, V578, W585, L587, I639, S641, G643, S674; and/or
ii) Q334, K376, H378, T381, V383, E390, E391, D392, D394, D433, E434, R439, S446, L452, G458, K466, S470, R471, V483, L486, R489, D500, F501, E507, I508, K512, R541, N564, D580, R583, E589; and/or

iii) D20, L21, E23, A28, R29, L303, G304, Q305, K325, R336, K377, K380, K422, T432, T435, K437, T438, E440, A441, Q443, H444, Q468, Y487, S488, L491, G494, K496, H497, L498, L504, G506, V517, D560, Q561, R562, T588, Q592, R593 of the native FVIII molecule

is substituted with a cysteine amino acid residue.

In another embodiment the invention is related to a factor VIII analogue wherein at least N-glycosylation site is introduced starting at a position selected from:

i) D27, F276, F306, L307, Y323, W382, H384, Y385, E389, W393, R427, M429, A430, I442, E445, I448, E456, V457, A469, T481, D482, K499, T514, E518, V537, N538, A544, G546, N572, F576, V578, W585, L587, I639, S641, G643, S674; and/or

ii) Q334, K376, T381, V383, E390, E391, D392, D394, D433, E434, R439, S446, L452, G458, K466, L486, R489, E507, I508, K512, R541, N564, D580, R583, E589; and/or

iii) D20, L21, A28, L303, G304, Q305, K325, R336, K380, K422, T432, T435, K437, T438, E440, A441, Q443, H444, Q468, Y487, S488, G494, K496, H497, L498, G506, V517, D560, Q561, R562, T588, Q592, R593 of the native FVIII molecule

In one embodiment the FVIII analogue comprises a Cys in at least one of position 377; 435; 488; 496 or 504.

In another embodiment the FVIII analogue comprises an Asn in position 433 or position 486 or in both.

In a further embodiment the FVIII analogue comprises an Asn in position 435 and a Thr or Ser in position 437.

In a still further embodiment the FVIII analogue comprises an Asn in position 488 and a Thr or Ser in position 490.

In a further embodiment the FVIII analogue comprises an Asn in position 496 and a Thr or Ser in position 498.

The Factor VIII analogues according to the present invention may comprise up three, up to two or a single amino acid substitution compared to the native human FVIII molecule.

The FVIII molecule may be the full length molecule or may lack part of or the whole B-domain.

In one embodiment of the present invention the FVIII analogue lacks at least 30% of the natural B-domain. In another embodiment the FVIII analogue lacks at least 50% of the natural B-domain and in a still further embodiment the FVIII analogue lacks from about 75 to about 85% of the natural B-domain or from about 85 to about 95% of the natural B-domain.

The FVIII analogue may also lack the whole B-domain.

In one embodiment the amino acid sequence from residue serine(S) in position 750 to cysteine (C) in position 1636 in the B-domain are deleted.

In another embodiment the amino acid sequence from residue threonine (T) in position 760 to asparagine (N) in position 1639 in the B-domain are deleted.

In a further aspect the present invention is related to a pharmaceutical formulation comprising the FVIII analogue according to the invention

In a further aspect the invention is related to a method for treatment of haemophilia patients by administration of a pharmaceutical formulation comprising a suitable amount of the FVIII analogue according to the present invention together with a pharmaceutically an acceptable carrier to a patient in need of such treatment.

In a further embodiment the FVIII formulation is administrated at least once per week.

In a further embodiment the FVIII formulation is administrated only once per week.

### DETAILED DESCRIPTION OF THE INVENTION

The human factor VIII gene was isolated and expressed in mammalian cells (Toole, J. J., et al., Nature 312:342-347 and U.S. Pat. No. 4,757,006) and the amino acid sequence was deduced from cDNA. U.S. Pat. No. 4,965,199 discloses a recombinant DNA method for producing factor VIII in mammalian host cells and purification of human factor VIII. U.S. Pat. No. 4,868,112 discloses modifying of human factor VIII to delete part or all of the B domain and U.S. Pat. No. 5,004,803 discloses replacement of the human factor VIII B domain with the human factor V B domain.

The amino acid sequence of the B and part of the A2 domains of porcine factor VIII was reported by Toole, J. J., et al., Proc. Natl. Acad. Sci. USA 83:5939-5942 (1986). The cDNA sequence encoding the complete A2 domain of porcine factor VIII and predicted amino acid sequence and hybrid human/porcine factor VIII is disclosed in US Pat. No. 5,364,771. WO 94/11503 discloses the nucleotide and corresponding amino acid sequences of the A1 and A2 domains of porcine factor VIII and a chimeric factor VIII with porcine A1 and/or A2 domains substituted for the corresponding human domains. U.S. Pat. No. 5,859,204 discloses the porcine cDNA and deduced amino acid sequences.

The present treatment of haemophilia with FVIII normally includes around three weekly injections supplemented with injections on a need basis, e.g. before tooth extractions or surgery. It is the purpose of the present invention to develop new FVIII analogues which only have to be injected once per week or less. FVIII circulates as an inactive proform and is only converted in the active form FVIIIa when a bleeding is to be arrested. Thus one way to accomplish a prophylactic FVIII treatment based on one weekly injection is to increase the circulation time of FVIII in the blood stream of the patient. In this way there will always be a certain level of inactive FVIII ready to be activated to ensure normal blood clotting conditions in the patient at any time.

The FVIII analogues according to the present invention are modified in the A1 and/or A2 domain by substituting of one or more of the natural amino acid residues with another amino acid residue which will create on or more N-glycosylation sites and/or by substituting one or more of the natural amino acid residues in these domains with a Cys residue. Thus the FVIII analogues may comprise one or more substitutions creating one or more N-glycosylation sites combined with insertion of one or more Cys residues instead of the natural amino acid residue in that position in the molecule.

Methods for introducing of mutations in a polypeptide are well established and are further exemplified in the examples.

The inserted cysteine residue may be modified by attachment of a chemical group. The modification of the inserted cysteine residue may be a) a mixed disulfide bond-formation with e.g. glutathione (γ-glutamylcysteinylglycine), γ-glutamylcysteine, or cysteine during or after synthesis of the FVIII polypeptide or b) in vitro modification of the inserted cysteine residue using thiol-specific chemistry as known to people skilled in the art.

The SH-group of cysteine residues represents a suitable chemical structure for derivatization since specific chemical reactions can be carried on this group without affecting other parts of the Factor VIII molecule. It is thus possible to couple side-chains onto this group, thereby obtaining Factor VIII derivatives with prolonged half-life compared to the non-derivatized molecule. Examples of such side-chain structures are: polyethylene glycols (PEGs), fatty acids, and carbohydrates. The specific chemical coupling is mediated by an appropriate reactive moiety linked to the side-chain structure that exhibit high selectivity towards labelling of free SH-groups. Commonly used functional groups for cysteine-directed chemical attachment include maleimide, vinylsulfone, iodoacetamide, and orthopyridyl disulfide.

The conjugation of a cysteine amino acid residue with the chemical group includes but are not limited to covalent attachment of polyethylene glycol (PEG), monomethoxy-polyethylene glycol, dextran, poly-(N-vinyl pyrrolidone) polyethylene glycol, propylene glycol homopolymers, a polypropylene oxide/ethylene oxide co-polymer, polypropylene glycol, polyoxyethylated polyols (e.g., glycerol) and polyvinyl alcohol, colominic acids or other carbohydrate based polymers, polymers of amino acids, and biotin derivatives.

The chemical group will typically be a biocompatible, non-toxic, non-immunogenic and water-soluble polymer. Preferably the chemical group is water-soluble in all proportions.

Methods for attaching PEG groups to cysteine residues is described in Roberts, M. J. et al, Advanced Drug Delivery Reviews 54 (2002) 459-476.

Specific examples of activated PEG polymers particularly preferred for coupling to cysteine residues include the following linear PEGs: vinylsulfone-PEG (VS-PEG), such as vinylsulfone-mPEG (VS-mPEG); maleimide-PEG (MAL-PEG), such as MALEIMIDE-MPEG (MAL-mPEG) and orthopyridyl-disulfide-PEG (OPSS-PEG), such as orthopyridyl-disulfide-MPEG (OPSS-MPEG). Typically, such PEG or MPEG polymers will have a size of about 5 kDa, about 10 kD, about 12 kDa or about 20 kDa.

For conjugation of a chemical group to a cysteine residue (e.g. PEGylation) the FVIII analogue is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to 16 hours.

Non limiting examples of suitable chemical groups are dendrimer, polyalkylene oxide (PAO), polyalkylene glycol (PAG), polyethylene glycol (PEG), polypropylene glycol (PPG), branched PEGs, polyvinyl alcohol (PVA), poly-carboxylate, poly-vinylpyrolidone, polyethylene-co-maleic acid anhydride, polystyrene-co-maleic acid anhydride, dextran, carboxymethyl-dextran; serum protein binding-ligands, such as compounds which bind to albumin, such as fatty acids, C5-C24 fatty acid, aliphatic diacid (e.g. C5-C24), a structure (e.g. sialic acid derivatives or mimetics) which inhibits the glycans from binding to receptors (e.g. asialoglycoprotein receptor and mannose receptor), a small organic molecule containing moieties that under physiological conditions alters charge properties, such as carboxylic acids or amines, or neutral substituents that prevent glycan specific recognition such as smaller alkyl substituents (e.g., C1-C5 alkyl), a low molecular organic charged radical (e.g. C1-C25), which may contain one or more carboxylic acids, amines sulfonic, phosphonic acids, or combination thereof; a low molecular neutral hydrophilic molecule (e.g. C1-C25), such as cyclodextrin, or a polyethylene chain which may optionally branched; polyethyleneglycol with a average molecular weight of 2-40 KDa; a well defined precission polymer such as a dendrimer with an exact molecular mass ranging from about 700 to about 20.000 Da or from about 700 to about 10.000 Da; and a substantially non-immunogenic polypeptide such as albumin or an antibody or part of an antibody optionally containing a Fc-domain.

Examples of FVIII mutations in or spatially near the A2 domain LRP (484-509) binding site are listed in Table 1. Non limiting examples of amino acid residue substitutions according to the present invention is given in Table 1.

TABLE 1
(numbering is according to SEQ ID NO: 1).

| | Introduction of |
|---|---|
| Introduction of N-glycosylation sites | cysteine residues |
| Asp433→Asn | Lys377→Cys |
| Thr435→Asn and Lys437→(Thr or Ser) | Thr435→Cys |
| Leu486→Asn | Ser488→Cys |
| Ser488→Asn and Arg490→(Thr or Ser) | Lys496→Cys |
| Lys496→Asn and Leu498→(Thr or Ser) | Leu504→Cys |

### DEFINITIONS

“Factor VIII” or “FVIII” as used herein refers to a plasma glycoprotein that is a member of the intrinsic coagulation pathway and is essential to blood coagulation.

“Native FVIII” is the full length human FVIII molecule as shown in SEQ ID NO:1. The numbering of the amino acid residue position is according to SEQ ID NO:1 where the first N-terminal amino acid residue is number 1 and so on. Unless otherwise specified or indicated, as used herein factor VIII means any functional human factor VIII protein molecule in its normal role in coagulation, including any fragment, analogue and derivative thereof. The expression FVIII will include mature human FVIII and FVIII analogues lacking one or more domains or lacking parts of one or more domains from the human FVIII molecule in particular the B-domain. Subunits of factor VIII are the heavy and light chains of the protein. The heavy chain of factor VIII contains three domains A1, A2, and B and the light chain of factor VIII likewise contains three domains A3, C1, and C2. Factor VIII is synthesized as an approximately 300 kDa single chain protein with the sequence A1-A2-B-A3-C1-C2-COO—H.

Unless otherwise specified, factor VIII domains include the following amino acid residues: A1 being the region from residue Ala1 to residue Arg372; A2 being the region from residue Ser373 to residue Arg740; B being the region from residue Ser741 to residue Arg1648; A3 being the region from residue Ser1690 to residue Ile2032; C1 being the region from residue Arg2033 to residue Asn2172; and C2 being the region from residue Ser2173 to residue Tyr2332. The A3-C1-C2 sequence includes residues Ser1690-Tyr2332. The remaining sequence, residues Glu1649-Arg1689, is usually referred to as the factor VIII light chain activation peptide.

A “B-domain deleted factor VIII” is a factor VIII molecule which lacks part of or the whole native B-domain. B-domain deleted factor VIII is well known and disclosed in U.S. Pat. No. 4,657,894; U.S. Pat. No. 4,749,780; U.S. Pat. No. 5,661,008; U.S. Pat. No. 4,868,112; EP patent No. 150,735 and EP patent No. 294,910.

“FVIII half-life” refers to the half-life of factor VIII in blood circulation, as determined in animals such as mice or in human, as determined by pharmacokinetics by standard procedures known to people skilled in the art. Human factor VIII has a half-life of about 12-14 hours.

A “FVIII clearance site” is defined as a region on the FVIII molecule that is recognized by the physiological machinery responsible for degradation of the protein. Included are the above-mentioned LRP and HSPG recognition sites.

A “disrupted clearance site” is defined as a clearance site on the FVIII molecule that exhibits reduced binding to its cognate receptor or interaction partner as a result of above-mentioned modification.

“FVIII activity” is defined as the ability to function in the coagulation cascade, induce the formation of FXa via interaction with FIXa on an activated platelet, and support the formation of a blood clot. The activity can be assessed in vitro by techniques such as clot analysis, as described in e.g. Manucci and Tripodi, “Factor VIII clotting activity”. E.C.A.T. assay procedures, London: Kluwer Academic Publishers, 1999; endogenous thrombin potential analysis, as described in Hemker et al., “The thrombogram: monitoring thrombin generation in platelet-rich plasma.”, Thrombosis and haemostasis, vol. 83:589-591; and other techniques known to people skilled in the art.

With the term a “factor VIII activity being substantially the same as the activity of activated human factor VIII” is meant a FVIII activity being at least 20%, at least 30%, at least 40%, at least 50%, at least 60%, at least 70%, at least 80%, at least 90% such as at least 100% of that of human FVIII. The FVIII activity is in particular about 50 to about 75%, about 75 to about 85%, about 85 to about 95% and even more than 100% of that of human FVIII.

“Prolonged FVIII” means a FVIII compound that circulates in a patient for an extended period of time following administration as compared to the native human FVIII.

An “N-glycosylation site” has the sequence N-Xaa-S/T, wherein Xaa is any amino acid residue except proline, N is asparagine and S/T is either serine or threonine, preferably threonine.

The term “inserted amino residue” is intended to include both a substitution of a natural amino acid residue with another amino acid residue, which is not normally found in that position in the native FVIII molecule, and an addition of an amino acid residue to the native human FVIII molecule. The addition of an amino acid residue may be either between two existing amino acid residues or at the N- or C-terminal end of the native FVIII molecule.

The term “LRP binding affinity”, as used herein, means the strength of the binding of FVIII polypeptide to human LRP. The affinity of a FVIII polypeptide is measured by the dissociation constant K_{d} measured by well known methods in the art, such as the Biacore technology (see example 7). The LRP binding affinity is preferably reduced by a at least a factor 2, such as at least a factor 3, e.g. at least a factor 4, such as at least a factor 5, e.g. at least a factor 6, such as at least a factor 7, e.g. at least a factor 8, such as at least a factor 9, e.g. at least a factor 10 of that of human factor VIII.

The term “PEGylated FVIII” means FVIII having a PEG molecule conjugated to the FVIII molecule. The term “cysteine-PEGylated FVIII” means FVIII having a PEG molecule conjugated to a sulfhydryl group of a cysteine introduced in FVIII molecule. The term “glycoPEGylated FVIII” means FVIII having a PEG molecule conjugated to a glycan structure on the FVIII molecule.

The terminology for amino acid substitutions used is as follows. The first letter represents the amino acid residue naturally present at a position of human FVIII. The following number represents the position in human FVIII. The second letter represent the different amino acid substituting for (replacing) the natural amino acid. An example is K377C, where a lysine at position 377 of human FVIII is replaced by a cysteine.

In the present context the three-letter or one-letter indications of the amino acids have been used in their conventional meaning as indicated in table 2. Unless indicated explicitly, the amino acids mentioned herein are L-amino acids. Further, the left and right ends of an amino acid sequence of a peptide are, respectively, the N- and C-termini unless otherwise specified.

TABLE 2
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The FVIII analogues may be produced by means of recombinant nucleic acid techniques. In general, a cloned human nucleic acid sequence is modified to encode the desired FVIII analogue and is then inserted into an expression vector, which is in turn transformed or transfected into host cells. Higher eukaryotic cells, in particular cultured mammalian cells, are preferred as host cells. The complete nucleotide and amino acid sequences for human FVIII is known, see U.S. Pat. No. 4,965,199 where the cloning and expression of recombinant human FVIII is described.

The amino acid sequence alterations may be accomplished by a variety of techniques. Modification of the nucleic acid sequence may be by site-specific mutagenesis. Techniques for site-specific mutagenesis are well known in the art and are described in, for example, Zoller and Smith (DNA 3:479-488, 1984) or “Splicing by extension overlap”, Horton et al., Gene 77, 1989, pp. 61-68. Thus, using the nucleotide and amino acid sequences of FVIII, one may introduce the alteration(s) of choice. Likewise, procedures for preparing a DNA construct using polymerase chain reaction using specific primers are well known to persons skilled in the art (cf. PCR Protocols, 1990, Academic Press, San Diego, Calif., USA).

The nucleic acid construct encoding the FVIII analogue of the invention may be of genomic or cDNA origin, for instance obtained by preparing a genomic or cDNA library and screening for DNA sequences coding for all or part of FVIII by hybridization using synthetic oligonucleotide probes in accordance with standard techniques (cf. Sambrook et al., Molecular Cloning: A Laboratory Manual, 2nd. Ed. Cold Spring Harbor Labora-tory, Cold Spring Harbor, N.Y., 1989).

The nucleic acid construct encoding the FVIII polypeptide analogue may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesised, e.g. in an automatic DNA synthesiser, purified, annealed, ligated and cloned in suitable vectors. The DNA sequences encoding the human FVIII polypeptides may also be prepared by polymerase chain reaction using specific primers, for instance as described in U.S. Pat. No. 4,683,202, Saiki et al., Science 239 (1988), 487-491, or Sambrook et al., supra.

Furthermore, the nucleic acid construct may be of mixed synthetic and genomic, mixed synthetic and cDNA or mixed genomic and cDNA origin prepared by ligating fragments of syn-thetic, genomic or cDNA origin (as appropriate), the fragments corresponding to various parts of the entire nucleic acid construct, in accordance with standard techniques.

The DNA sequences encoding the FVIII polypeptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the FVIII analogue is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

Expression vectors for use in expressing FVIII analogues will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell.

Examples of suitable promoters for directing the transcription of the DNA encoding the FVIII analogues in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

The DNA sequences encoding the FVIII analogue may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. Expression vectors may also contain a set of RNA splice sites located downstream from the promoter and upstream from the insertion site for the FVIII sequence itself. Preferred RNA splice sites may be obtained from adenovirus and/or immunoglobulin genes. Also contained in the expression vectors is a polyadenylation signal located down-stream of the insertion site. Particularly preferred polyadenylation signals include the early or late polyadenylation signal from SV40 (Kaufman and Sharp, ibid.), the polyadenylation signal from the adenovirus 5 EIb region, the human growth hormone gene terminator (DeNoto et al. Nucl. Acids Res. 9:3719-3730, 1981) or the polyadenylation signal from the human FVIII gene. The expression vectors may also include a noncoding viral leader sequence, such as the adenovirus 2 tripartite leader, located between the promoter and the RNA splice sites; and enhancer sequences, such as the SV40 enhancer.

To direct the FVIII analogue of the present invention into the secretory pathway of the host cells, a secretory signal sequence (also known as a leader sequence, prepro sequence or pre sequence) may be provided in the recombinant vector. The secretory signal sequence is joined to the DNA sequences encoding the FVIII analogues in the correct reading frame. Secretory signal sequences are commonly positioned 5′ to the DNA sequence encoding the peptide. The secretory signal sequence may be that, normally associated with the protein or may be from a gene encoding another secreted protein.

The procedures used to ligate the DNA sequences coding for the FVIII analogues, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973) or electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker may be an amplifiable selectable marker. A preferred amplifiable selectable marker is a dihydrofolate reductase (DHFR) sequence. Selectable markers are reviewed by Thilly (Mammalian Cell Technology, Butterworth Publishers, Stoneham, Mass., incorporated herein by reference). The person skilled in the art will easily be able to choose suitable selectable markers.

Selectable markers may be introduced into the cell on a separate plasmid at the same time as the gene of interest, or they may be introduced on the same plasmid. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the FVIII analogues. Media generally include a carbon source, a nitrogen source, essential amino acids, essential sugars, vitamins, salts, phospholipids, protein and growth factors. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the FVIII analogue.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk-ts31 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk-ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and CHO-DUKX cells (Urlaub and Chasin, Proc. Natl. Acad. Sci. USA 77:4216-4220, 1980).

FVIII analogues of the invention are recovered from cell culture medium and can then be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). Preferably, they may be purified by affinity chromatography on an anti-FVIII antibody column. Additional purification may be achieved by conventional chemical purification means, such as high performance liquid chromatography. Other methods of purification are known in the art, and may be applied to the purification of the novel FVIII polypeptides described herein (see, for example, Scopes, R., Protein Purification, Springer-Verlag, N.Y., 1982).

For therapeutic purposes it is preferred that the FVIII analogue is purified to at least about 90 to 95% homogeneity, preferably to at least about 98% homogeneity. Purity may be assessed by e.g. gel electrophoresis and amino-terminal amino acid sequencing.

In another aspect the present invention is related to a pharmaceutical formulation comprising a FVIII analogue in a dried form, whereto the physician or the patient adds solvents and/or diluents prior to use. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

In a further aspect the invention relates to a pharmaceutical formulation comprising an aqueous solution of a FVIII analogue and a buffer, wherein the FVIII analogue is present in a concentration from 0.01 mg/ml or above, and wherein said formulation has a pH from about 2.0 to about 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or DL isomer) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, or nithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or DL isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation comprises a surfactant. The surfactant may be a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)—derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or ginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyltrimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

It is possible that other ingredients may be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the FVIII compound in the form of a nasal or pulmonal spray. As a still further option, the pharmaceutical compositions containing the FVIII compound of the invention can also be adapted to transdermal administration, e.g. by needle-free injection or from a patch, optionally an iontophoretic patch, or transmucosal, e.g. buccal, administration.

Full Length Factor VIII has the following sequence
(SEQ ID NO: 1):
ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
TRQKQFNATTIPENDIEKTDPWFAHRTPMPKIQNVSSSDLLMLLRQSPTP
HGLSLSDLQEAKYETFSDDPSPGAIDSNNSLSEMTHFRPQLHHSGDMVFT
PESGLQLRLNEKLGTTAATELKKLDFKVSSTSNNLISTIPSDNLAAGTDN
TSSLGPPSMPVHYDSQLDTTLFGKKSSPLTESGGPLSLSEENNDSKLLES
GLMNSQESSWGKNVSSTESGRLFKGKRAHGPALLTKDNALFKVSISLLKT
NKTSNNSATNRKTHIDGPSLLIENSPSVWQNILESDTEFKKVTPLIHDRM
LMDKNATALRLNHMSNKTTSSKNMEMVQQKKEGPIPPDAQNPDMSFFKML
FLPESARWIQRTHGKNSLNSGQGPSPKQLVSLGPEKSVEGQNFLSEKNKV
VVGKGEFTKDVGLKEMVFPSSRNLFLTNLDNLHENNTHNQEKKIQEEIEK
KETLIQENVVLPQIHTVTGTKNFMKNLFLLSTRQNVEGSYDGAYAPVLQD
FRSLNDSTNRTKKHTAHFSKKGEEENLEGLGNQTKQIVEKYACTTRISPN
TSQQNFVTQRSKRALKQFRLPLEETELEKRIIVDDTSTQWSKNMKHLTPS
TLTQIDYNEKEKGAITQSPLSDCLTRSHSIPQANRSPLPIAKVSSFPSIR
PIYLTRVLFQDNSSHLPAASYRKKDSGVQESSHFLQGAKKNNLSLAILTL
EMTGDQREVGSLGTSATNSVTYKKVENTVLPKPDLPKTSGKVELLPKVHI
YQKDLFPTETSNGSPGHLDLVEGSLLQGTEGAIKWNEANRPGKVPFLRVA
TESSAKTPSKLLDPLAWDNHYGTQIPKEEWKSQEKSPEKTAFKKKDTILS
LNACESNHAIAAINEGQNKPEIEVTWAKQGRTERLCSQNPPVLKRHQREI
TRTTLQSDQEEIDYDDTISVEMKKEDFDIYDEDENQSPRSFQKKTRHYFI
AAVERLWDYGMSSSPHVLRNRAQSGSVPQFKKVVFQEFTDGSFTQPLYRG
ELNEHLGLLGPYIRAEVEDNIMVTFRNQASRPYSFYSSLISYEEDQRQGA
EPRKNFVKPNETKTYFWKVQHHMAPTKDEFDCKAWAYFSDVDLEKDVHSG
LIGPLLVCHTNTLNPAHGRQVTVQEFALFFTIFDETKSWYFTENMERNCR
APCNIQMEDPTFKENYRFHAINGYIMDTLPGLVMAQDQRIRWYLLSMGSN
ENIHSIHFSGHVFTVRKKEEYKMALYNLYPGVFETVEMLPSKAGIWRVEC
LIGEHLHAGMSTLFLVYSNKCQTPLGMASGHIRDFQITASGQYGQWAPKL
ARLHYSGSINAWSTKEPFSWIKVDLLAPMIIHGIKTQGARQKFSSLYISQ
FIIMYSLDGKKWQTYRGNSTGTLMVFFGNVDSSGIKHNIFNPPIIARYIR
LHPTHYSIRSTLRMELMGCDLNSCSMPLGMESKAISDAQITASSYFTNMF
ATWSPSKARLHLQGRSNAWRPQVNNPKEWLQVDFQKTMKVTGVTTQGVKS
LLTSMYVKEFLISSSQDGHQWTLFFQNGKVKVFQGNQDSFTPVVNSLDPP
LLTRYLRIHPQSWVHQIALRMEVLGCEAQDLY
Sequence F8-500 in pTT5 (without mutations) has
the following sequence (SEQ ID NO: 2):
gtacatttatattggctcatgtccaatatgaccgccatgttgacattgat
tattgactagttattaatagtaatcaattacggggtcattagttcatagc
ccatatatggagttccgcgttacataacttacggtaaatggcccgcctgg
ctgaccgcccaacgacccccgcccattgacgtcaataatgacgtatgttc
ccatagtaacgccaatagggactttccattgacgtcaatgggtggagtat
ttacggtaaactgcccacttggcagtacatcaagtgtatcatatgccaag
tccgccccctattgacgtcaatgacggtaaatggcccgcctggcattatg
cccagtacatgaccttacgggactttcctacttggcagtacatctacgta
ttagtcatcgctattaccatggtgatgcggttttggcagtacaccaatgg
gcgtggatagcggtttgactcacggggatttccaagtctccaccccattg
acgtcaatgggagtttgttttggcaccaaaatcaacgggactttccaaaa
tgtcgtaataaccccgccccgttgacgcaaatgggcggtaggcgtgtacg
gtgggaggtctatataagcagagctcgtttagtgaaccgtcagatcctca
ctctcttccgcatcgctgtctgcgagggccagctgttgggctcgcggttg
aggacaaactcttcgcggtctttccagtactcttggatcggaaacccgtc
ggcctccgaacggtactccgccaccgagggacctgagcgagtccgcatcg
accggatcggaaaacctctcgagaaaggcgtctaaccagtcacagtcgca
aggtaggctgagcaccgtggcgggcggcagcgggtggcggtcggggttgt
ttctggcggaggtgctgctgatgatgtaattaaagtaggcggtcttgaga
cggcggatggtcgaggtgaggtgtggcaggcttgagatccagctgttggg
gtgagtactccctctcaaaagcgggcattacttctgcgctaagattgtca
gtttccaaaaacgaggaggatttgatattcacctggcccgatctggccat
acacttgagtgacaatgacatccactttgcctttctctccacaggtgtcc
actcccaggtccaagtttaaacggatctctagcgaattccctctagaggg
cccgtttctgctagcaagcttgctagcggccgcgtttaactggtaagttt
agtctttttgtcttttatttcaggtcccggatcgaagcttggctgcaggt
cgacgccaccatggaaatagagctctccacctgcttctttctgtgccttt
tgcgattctgctttagtgccaccagaagatactacctgggtgcagtggaa
ctgtcatgggactatatgcaaagtgatctcggtgagctgcctgtggacgc
aagatttcctcctagagtgccaaaatcttttccattcaacacctcagtcg
tgtacaaaaagactctgtttgtagaattcacggatcaccttttcaacatc
gctaagccaaggccaccctggatgggtctgctaggtcctaccatccaggc
tgaggtttatgatacagtggtcattacacttaagaacatggcttcccatc
ctgtcagtcttcatgctgttggtgtatcctactggaaagcttctgaggga
gctgaatatgatgatcagaccagtcaaagggagaaagaagatgataaagt
cttccctggtggaagccatacatatgtctggcaggtcctgaaagagaatg
gtccaatggcctctgacccactgtgccttacctactcatatctttctcat
gtggacctggtaaaagacttgaattcaggcctcattggagccctactagt
atgtagagaagggagtctggccaaggaaaagacacagaccttgcacaaat
ttatactactttttgctgtatttgatgaagggaaaagttggcactcagaa
acaaagaactccttgatgcaggatagggatgctgcatctgctcgggcctg
gcctaaaatgcacacagtcaatggttatgtaaacaggtctctgccaggtc
tgattggatgccacaggaaatcagtctattggcatgtgattggaatgggc
accactcctgaagtgcactcaatattcctcgaaggtcacacatttcttgt
gaggaaccatcgccaggcgtccttggaaatctcgccaataactttcctta
ctgctcaaacactcttgatggaccttggacagtttctactgttttgtcat
atctcttcccaccaacatgatggcatggaagcttatgtcaaagtagacag
ctgtccagaggaaccccaactacgaatgaaaaataatgaagaagcggaag
actatgatgatgatcttactgattctgaaatggatgtggtcaggtttgat
gatgacaactctccttcctttatccaaattcgctcagttgccaagaagca
tcctaaaacttgggtacattacattgctgctgaagaggaggactgggact
atgctcccttagtcctcgcccccgatgacagaagttataaaagtcaatat
ttgaacaatggccctcagcggattggtaggaagtacaaaaaagtccgatt
tatggcatacacagatgaaacctttaagactcgtgaagctattcagcatg
aatcaggaatcttgggacctttactttatggggaagttggagacacactg
ttgattatatttaagaatcaagcaagcagaccatataacatctaccctca
cggaatcactgatgtccgtcctttgtattcaaggagattaccaaaaggtg
taaaacatttgaaggattttccaattctgccaggagaaatattcaaatat
aaatggacagtgactgtagaagatgggccaactaaatcagatcctcggtg
cctgacccgctattactctagtttcgttaatatggagagagatctagctt
caggactcattggccctctcctcatctgctacaaagaatctgtagatcaa
agaggaaaccagataatgtcagacaagaggaatgtcatcctgttttctgt
atttgatgagaaccgaagctggtacctcacagagaatatacaacgctttc
tccccaatccagctggagtgcagcttgaggatccagagttccaagcctcc
aacatcatgcacagcatcaatggctatgtttttgatagtttgcagttgtc
agtttgtttgcatgaggtggcatactggtacattctaagcattggagcac
agactgacttcctttctgtcttcttctctggatataccttcaaacacaaa
atggtctatgaagacacactcaccctattcccattctcaggagaaactgt
cttcatgtcgatggaaaacccaggtctatggattctggggtgccacaact
cagactttcggaacagaggcatgaccgccttactgaaggtttctagttgt
gacaagaacactggtgattattacgaggacagttatgaagatatttcagc
atacttgctgagtaaaaacaatgccattgaaccaagaagcttctcccaga
attcgcgacaccctagccaaaacccaccggtcttgaaacgccatcaacgg
gagatcactcgtactactcttcagtctgatcaagaggaaattgactatga
tgataccatatcagttgaaatgaagaaggaagattttgacatttatgatg
aggatgaaaatcagagcccccgcagctttcaaaagaaaacacgacactat
tttattgctgcagtggagaggctctgggattatgggatgagtagctcccc
acatgttctaagaaacagggctcagagtggcagtgtccctcagttcaaga
aagttgttttccaggaatttactgatggctcctttactcagcccttatac
cgtggagaactaaatgaacatttgggactcctggggccatatataagagc
agaagttgaagataatatcatggtaactttcagaaatcaggcctctcgtc
cctattccttctattctagccttatttcttatgaggaagatcagaggcaa
ggagcagaacctagaaaaaactttgtcaagcctaatgaaaccaaaactta
cttttggaaagtgcaacatcatatggcacccactaaagatgagtttgact
gcaaagcctgggcttatttctctgatgttgacctggaaaaagatgtgcac
tcaggcctgattggaccccttctggtctgccacactaacacactgaaccc
tgctcatgggagacaagtgacagtacaggaatttgctctgtttttcacca
tctttgatgagaccaaaagctggtacttcactgaaaatatggaaagaaac
tgcagggctccctgcaatatccagatggaagatcccacttttaaagagaa
ttatcgcttccatgcaatcaatggctacataatggatacactacctggct
tagtaatggctcaggatcaaaggattcgatggtatctgctcagcatgggc
agcaatgaaaacatccattctattcatttcagtggacatgtgttcactgt
acgaaaaaaagaggagtataaaatggcactgtacaatctctatccaggtg
tttttgagacagtggaaatgttaccatccaaagctggaatttggcgggtg
gaatgccttattggcgagcatctacatgctgggatgagcacactttttct
ggtgtacagcaataagtgtcagactcccctgggaatggcttctggacaca
ttagagattttcagattacagcttcaggacaatatggacagtgggcccca
aagctggccagacttcattattccggatcaatcaatgcctggagcaccaa
ggagcccttttcttggatcaaggtggatctgttggcaccaatgattattc
acggcatcaagacccagggtgcccgtcagaagttctccagcctctacatc
tctcagtttatcatcatgtatagtcttgatgggaagaagtggcagactta
tcgaggaaattccactggaaccttaatggtcttctttggcaatgtggatt
catctgggataaaacacaatatttttaaccctccaattattgctcgatac
atccgtttgcacccaactcattatagcattcgcagcactcttcgcatgga
gttgatgggctgtgatttaaatagttgcagcatgccattgggaatggaga
gtaaagcaatatcagatgcacagattactgcttcatcctactttaccaat
atgtttgccacctggtctccttcaaaagctcgacttcacctccaagggag
gagtaatgcctggagacctcaggtgaataatccaaaagagtggctgcaag
tggacttccagaagacaatgaaagtcacaggagtaactactcagggagta
aaatctctgcttaccagcatgtatgtgaaggagttcctcatctccagcag
tcaagatggccatcagtggactctcttttttcagaatggcaaagtaaagg
tttttcagggaaatcaagactccttcacacctgtggtgaactctctagac
ccaccgttactgactcgctaccttcgaattcacccccagagttgggtgca
ccagattgccctgaggatggaggttctgggctgcgaggcacaggacctct
actgaggatccccaaacgctagagcggccgctcgaggccggcaaggccgg
atcccccgacctcgacctctggctaataaaggaaatttattttcattgca
atagtgtgttggaattttttgtgtctctcactcggaaggacatatgggag
ggcaaatcatttggtcgagatccctcggagatctctagctagaggatcga
tccccgccccggacgaactaaacctgactacgacatctctgccccttctt
cgcggggcagtgcatgtaatcccttcagttggttggtacaacttgccaac
tgaaccctaaacgggtagcatatgcttcccgggtagtagtatatactatc
cagactaaccctaattcaatagcatatgttacccaacgggaagcatatgc
tatcgaattagggttagtaaaagggtcctaaggaacagcgatgtaggtgg
gcgggccaagataggggcgcgattgctgcgatctggaggacaaattacac
acacttgcgcctgagcgccaagcacagggttgttggtcctcatattcacg
aggtcgctgagagcacggtgggctaatgttgccatgggtagcatatacta
cccaaatatctggatagcatatgctatcctaatctatatctgggtagcat
aggctatcctaatctatatctgggtagcatatgctatcctaatctatatc
tgggtagtatatgctatcctaatttatatctgggtagcataggctatcct
aatctatatctgggtagcatatgctatcctaatctatatctgggtagtat
atgctatcctaatctgtatccgggtagcatatgctatcctaatagagatt
agggtagtatatgctatcctaatttatatctgggtagcatatactaccca
aatatctggatagcatatgctatcctaatctatatctgggtagcatatgc
tatcctaatctatatctgggtagcataggctatcctaatctatatctggg
tagcatatgctatcctaatctatatctgggtagtatatgctatcctaatt
tatatctgggtagcataggctatcctaatctatatctgggtagcatatgc
tatcctaatctatatctgggtagtatatgctatcctaatctgtatccggg
tagcatatgctatcctcatgataagctgtcaaacatgagaattaattctt
gaagacgaaagggcctcgtgatacgcctatttttataggttaatgtcatg
ataataatggtttcttagacgtcaggtggcacttttcggggaaatgtgcg
cggaacccctatttgtttatttttctaaatacattcaaatatgtatccgc
tcatgagacaataaccctgataaatgcttcaataatattgaaaaaggaag
agtatgagtattcaacatttccgtgtcgcccttattcccttttttgcggc
attttgccttcctgtttttgctcacccagaaacgctggtgaaagtaaaag
atgctgaagatcagttgggtgcacgagtgggttacatcgaactggatctc
aacagcggtaagatccttgagagttttcgccccgaagaacgttttccaat
gatgagcacttttaaagttctgctatgtggcgcggtattatcccgtgttg
acgccgggcaagagcaactcggtcgccgcatacactattctcagaatgac
ttggttgagtactcaccagtcacagaaaagcatcttacggatggcatgac
agtaagagaattatgcagtgctgccataaccatgagtgataacactgcgg
ccaacttacttctgacaacgatcggaggaccgaaggagctaaccgctttt
ttgcacaacatgggggatcatgtaactcgccttgatcgttgggaaccgga
gctgaatgaagccataccaaacgacgagcgtgacaccacgatgcctgcag
caatggcaacaacgttgcgcaaactattaactggcgaactacttactcta
gcttcccggcaacaattaatagactggatggaggcggataaagttgcagg
accacttctgcgctcggcccttccggctggctggtttattgctgataaat
ctggagccggtgagcgtgggtctcgcggtatcattgcagcactggggcca
gatggtaagccctcccgtatcgtagttatctacacgacggggagtcaggc
aactatggatgaacgaaatagacagatcgctgagataggtgcctcactga
ttaagcattggtaactgtcagaccaagtttactcatatatactttagatt
gatttaaaacttcatttttaatttaaaaggatctaggtgaagatcctttt
tgataatctcatgaccaaaatcccttaacgtgagttttcgttccactgag
cgtcagaccccgtagaaaagatcaaaggatcttcttgagatccttttttt
ctgcgcgtaatctgctgcttgcaaacaaaaaaaccaccgctaccagcggt
ggtttgtttgccggatcaagagctaccaactctttttccgaaggtaactg
gcttcagcagagcgcagataccaaatactgttcttctagtgtagccgtag
ttaggccaccacttcaagaactctgtagcaccgcctacatacctcgctct
gctaatcctgttaccagtggctgctgccagtggcgataagtcgtgtctta
ccgggttggactcaagacgatagttaccggataaggcgcagcggtcgggc
tgaacggggggttcgtgcacacagcccagcttggagcgaacgacctacac
cgaactgagatacctacagcgtgagctatgagaaagcgccacgcttcccg
aagggagaaaggcggacaggtatccggtaagcggcagggtcggaacagga
gagcgcacgagggagcttccagggggaaacgcctggtatctttatagtcc
tgtcgggtttcgccacctctgacttgagcgtcgatttttgtgatgctcgt
caggggggcggagcctatggaaaaacgccagcaacgcggcctttttacgg
ttcctggccttttgctggccttttgctcacatgttctttcctgcgttatc
ccctgattctgtggataaccgtattaccgcctttgagtgagctgataccg
ctcgccgcagccgaacgaccgagcgcagcgagtcagtgagcgaggaagc.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

### Example 1

### Construction of B-Domain Deleted-FVIII Mutants

In order to introduce N-glycosylation sites or cysteine residues in, or spatially near, the A2 domain LRP binding site, an 1828 bp FVIII Heavy-Chain fragment was subcloned from F8-500 (SEQ ID NO:2) (coding for a B-domain deleted FVIII) in pTT5 into pBluescript II SK+ using the restriction enzymes SalI and KpnI. All mutations were introduced using the QuikChange® Site-Directed Mutagenesis Kit (Stratagene, La Jolla, Calif.). Complementary primers (denoted Primer 1 & Primer 2) harbouring the desired nucleotide changes were designed and are shown in table 3. Sequence verified mutations were subcloned from pBluescript II SK+ back to F8-500 in pTT5 using the restriction enzymes SalI and KpnI. Verifications of the mutations in pTT5 were done by sequencing.

TABLE 3

Complementary primers used in the mutagenesis
of B-domain deleted FV III.

| | | |
|---|---|---|
| Mutation | Primer 1 (5′-3′) | Primer 2 (5′-3′) |

| | | |
|---|---|---|
| Residue | CGC TCA GTT GCC | CCC AAG TTT TAG GAT GAC |
| 377K→C | AAG TGT CAT CCT | ACT TGG CAA CTG AGC G |
| | AAA ACT TGG G | (SEQ ID NO: 4) |
| | (SEQ ID NO: 3) | |
| Residue | TTT ATG GCA TAC | GTC TTA AAG GTT TCA TTT |
| 433D→N | ACA AAT GAA ACC | GTG TAT GCC ATA AA |
| | TTT AAG AC | (SEQ ID NO: 6) |
| | (SEQ ID NO: 5) | |
| Residue | GGC ATA CAC AGA | GAA TAG CTT CAC GAG TCT |
| 435T→C | TGA ATG CTT TAA | TAA AGC ATT CAT CTG TGT |
| | GAC TCG TGA AGC | ATG CC |
| | TAT TC | (SEQ ID NO: 8) |
| | (SEQ ID NO: 7) | |
| Residue | GGC ATA CAC AGA | CTG AAT AGC TTC ACG AGT |
| 435T→N & | TGA AAA CTT TAC | CGT AAA GTT TTC ATC TGT |
| Residue | GAC TCG TGA AGC | GTA TGC C |
| 437K→T | TAT TCA G | (SEQ ID NO: 10) |
| | (SEQ ID NO: 9) | |
| Residue | GAT GTC CGT CCT | CTT TTG GTA ATC TCC TTG |
| 486L→N | AAT TAT TCA AGG | AAT AAT TAG GAC GGA CAT |
| | AGA TTA CCA AAA | C |
| | G | (SEQ ID NO: 12) |
| | (SEQ ID NO: 11) | |
| Residue | GAT GTC CGT CCT | CAC CTT TTG GTA ATC TCC |
| 488S→C | TTG TAT TGT AGG | TAC AAT ACA AAG GAC GGA |
| | AGA TTA CCA AAA | CAT C |
| | GGT G | (SEQ ID NO: 14) |
| | (SEQ ID NO: 13) | |
| Residue | GTC CGT CCT TTG | CAC CTT TTG GTA ATG TCC |
| 488S→N & | TAT AAC AGG ACA | TGT TAT ACA AAG GAC GGA |
| Residue | TTA CCA AAA GGT | C |
| 490R→T | G | (SEQ ID NO: 16) |
| | (SEQ ID NO: 15) | |
| Residue | GAT TAC CAA AAG | GAA TTG GAA AAT CCT TCA |
| 496K→C | GTG TAT GCC ATT | AAT GGC ATA CAC CTT TTG |
| | TGA AGG ATT TTC | GTA ATC |
| | CAA TTC | (SEQ ID NO: 18) |
| | (SEQ ID NO: 17) | |
| Residue | CCA AAA GGT GTA | GCA GAA TTG GAA AAT CCT |
| 496K→N & | AAT CAT TCG AAG | TCG AAT GAT TTA CAC CTT |
| Residue | GAT TTT CCA ATT | TTG G |
| 498L→S | CTG C | (SEQ ID NO: 20) |
| | (SEQ ID NO: 19) | |
| Residue | GAA GGA TTT TCC | CAT TTA TAT TTG AAT ATT |
| 504L→C | AAT TTG CCC AGG | TCT CCT GGG CAA ATT GGA |
| | AGA AAT ATT CAA | AAA TCC TTC |
| | ATA TAA ATG | (SEQ ID NO: 22) |
| | (SEQ ID NO: 21) | |

### Construction of B-Domain Deleted-FVIII Mutant (Residue 377K→C)

20 ng dsDNA template (pBluescript II SK+ with 1828 bp SalI-KpnI FVIII Heavy-Chain fragment) were combined with 2.5 μl 10× reaction buffer (Stratagene), 0.5 μl dNTP mix (Stratagene), 0.5 μl PFUTurbo DNA polymerase (2.5 U/μl) (Stratagene), 62.5 ng Primer 1 CGC TCA GTT GCC AAG TGT CAT CCT AAA ACT TGG G (SEQ ID NO:3), and 62.5 ng Primer 2 CCC AAG TTT TAG GAT GAC ACT TGG CAA CTG AGC G (SEQ ID NO:4). The reaction was incubated at 95° C. C for 30 seconds and subsequently cycled (18 cycles) at 95° C. for 30 seconds, 55° C. for 1 minute, and 68° C. for 4 minutes in a DNA Engine thermal cycler (MJResearch, Waltham, Mass.). Amplification products were digested with DpnI and XL1-Blue supercompetent cells were transformed following the instructions of the manufacturer (Stratagene). The introduced mutations were verified by sequencing the 1828 bp SalI-KpnI FVIII Heavy chain fragment in pBluescriptII SK+ (MWG DNA sequencing service). After subcloning the 1828 bp SalI-KpnI FVIIII Heavy chain fragment back to pTT5 the sequence verification was repeated.

### Example 2

### Cysteine-Directed PEGylation of BDD FVIII Cys Mutants

Materials—Reduced and oxidized glutathione (GSH and GSSG, respectively) were purchased from Sigma. PEG5k-maleimide (2E2M0H01) was purchased from Nektar Therapeutics (Huntsville, Ala.).

Concentration determination—The concentration of GSSG in stock solutions was determined from its absorption at 248 nm using an extinction coefficient of 381 M⁻¹ cm⁻¹ (Chau and Nelson, 1991). The concentration of GSH was determined using Ellman's reagent (5,5′-dithiobis(2-nitrobenzoic acid)) and 14150 M⁻¹ cm⁻¹ as the molar extinction coefficient of 2-nitro-5-thiobenzoic acid at 412 nm (Riddles et al., 1979).

Cloning and expression of glutaredoxins—The DNA coding sequence for Escherichia coli glutaredoxin 2 (Grx2; (Vlamis-Gardikas et al., 1997)) was amplified by PCR using Expand High Fidelity PCR system (Roche Diagnostics Corporation, Indianapolis, Ind.) according to manufacturer's recommendations and primers oHOJ98-f and oHOJ98-r introducing flanking NdeI and XhoI restriction sites (primer sequences are listed in Table 4). Genomic template DNA for the PCR reaction was prepared from E. coli according to published procedures (Grimberg et al., 1989). The purified PCR product was cut with NdeI and XhoI and then ligated into the corresponding site of pET-24a(+) (Novagen) to give pHOJ294. Since the stop codon was provided by the vector, the gene was equipped with a 3′ vector-derived extension encoding a C-terminal LEHHHHHH affinity tag. The correct identity of the cloned sequence was verified by DNA sequencing.

For expression, pHOJ294 was introduced into chemical competent BL21 (DE3) cells (Stratagene, La Jolla, Calif.). Fresh overnight transformants were inoculated into 500 ml terrific broth ((Sambrook et al., 1989)) and 30 μg/ml kanamycine to an initial OD₆₀₀ of 0.02. Cultures were grown at 37° C. in baffled flasks at 230 rpm to the mid-log phase (OD₆₀₀ 3-4) at which time the temperature was lowered to 25° C. and protein expression induced by 0.1 mM isopropyl-β-D-thiogalactopyranoside (ITPG). After overnight expression, cells were harvested by centrifugation, resuspended in 50 ml lysis buffer (50 mM potassium phosphate, 300 mM NaCl, pH 8.0), and lysed by three freeze-thaw cycles. The cleared lysate was loaded onto a 20-ml Ni-NTA Superflow (Qiagen GmbH, Hilden, Germany) column equilibrated with lysis buffer at a flow rate of 5 ml/min. After washing with lysis buffer, bound protein was eluted with a linear gradient from 0-200 mM imidazole in lysis buffer. Peak fractions were pooled, treated with 20 mM dithiothreitol for 20 min before extensive dialysis against 50 mM Tris-HCl, 2 mM EDTA, pH 8.0. The protein was stored at −80° C. and judged to be >90% pure by SDS-PAGE. Protein concentration was estimated by absorbance at 280 nm using an extinction coefficient of 21740 M⁻¹ cm⁻¹.

Selective reduction and PEG5k modification—The thiol modification procedure can be divided into two consecutive steps consisting of (A) a glutaredoxin-catalyzed selective reduction reaction where engineered cysteines are prepared for subsequent modification by selective reduction of mixed disulfides with low-molecular weight thiols, followed by (B) thiol-specific alkylation of liberated cysteines. In the first step, F8-500-T435C and F8-500-L504C were incubated for 2 hours at 30° C. in a total volume of 265 μl 20 mM imidazole, 150 mM NaCl, 10 mM CaCl₂, 10% glycerol, 0.02% Tween 80, pH 7.3 buffer containing 0.5 mM GSH, 10 μM GSSG, and 10 μM recombinant E. coli glutaredoxin 2 (Grx2). To remove low-molecular weight thiols before PEG5k-maleimide labeling, aliquots of the reaction mixtures (65 μl) were gelfiltrated on Pro-Spin spin columns (Catalog No CS-800; Princeton Separations, Adelphia, N.J.) equilibrated in 20 mM imidazole, 150 mM NaCl, 10 mM CaCl₂, 10% glycerol, 0.02% Tween 80, pH 7.3 buffer according to manufacturer's instructions. Free thiols were then modified by addition of PEG5k-maleimide (dissolved in water) to a final concentration of 0.25 mM. Thiol alkylation was allowed to proceed for 20 min at room temperature before dilution of the samples into reducing NuPAGE LDS sample buffer to quench the reaction and pre-pare for subsequent SDS-PAGE analysis (Invitrogen Life Technologies, Carlsbad, Calif.). To demonstrate the efficacy of the selective reduction reaction, two additional samples of F8-500-T435C and F8-500-L504C were treated as described above except that the initial incubation was performed in the absence of GSH, GSSG, and Grx2.

Samples of F8-500-T435C and F8-500-L504C were analyzed by reducing SDS-PAGE on a 4-12% Bis-Tris NuPAGE® gel (Invitrogen Life Technologies, Carlsbad, Calif.) run at 200 V for 60 min in MOPS buffer (Invitrogen Life Technologies, Carlsbad, Calif.) according to manufacturer's recommendations. The gel was washed with water and stained with Simply Blue™ SafeStain (Invitrogen Life Technologies, Carlsbad, Calif.) according to manufacturer's recommendations. It was found that the selective reduction and PEGylation resulted in the Heavy Chain being modified with a single PEG5k.

Table 4

DNA oligos used for construction of plasmid pHOJ294 expressing E. coli glutaredoxin 2 (Grx2). NdeI and XhoI restriction sites are shown in bold face.

TABLE 4

| | | | |
|---|---|---|---|
| Primer | Plasmid | Target | Sequence (5′→3′) |

| | | | |
|---|---|---|---|
| oHOJ98-f | pHOJ294 | Grx2 | GCCGCCGGCATATGAAGCTATA- |
| | | | CATTTACGATCACTGCCC |
| | | | (SEQ ID NO: 23) |
| oHOJ98-r | pHOJ294 | Grx2 | CCGCCGCCCTCGA- |
| | | | GAATCGCCATTGATGA- |
| | | | TAACAAATTGATTTGTG |
| | | | (SEQ ID NO: 24) |

### Example 3

### Transient Expression and Activity Testing of FVIII Mutants

Suspension adapted human embryonal kidney (HEK293F) cells (Freestyle, Invitrogen) were transfected with expression plasmids encoding wild-type BDD FVIII or mutant BDD FVIII per manufacturer's instructions. Briefly, 30 μg of plasmid was incubated 20 min with 40 l 293fectin (Invitrogen) and added to 3×10⁷ cells in a 125 ml Erlenmeyer flask. The transfected cells were incubated in a shaking incubator (37° C., 8% CO₂ and 125 rpm). Two days post-transfection, the cells were moved to a 27° C. shaking incubator. Four days post-transfection, the culture was centrifugated 1500×g for 5 min, and the cell pellet was discarded. The supernatant was stabilized by addition of imidazol pH 7.2 to a final concentration of 20 mM and Tween 80 to a final concentration of 0.02% and frozen in aliquots at −80° C. The yield of each mutant was determined by sandwich ELISA. Aliquots of stabilized and frozen medium were thawed and assayed with the Matched-Pair Antibody Set for ELISA of human Factor VIII antigen (Affinity Biologicals).

For activity testing, aliquots of stabilized and frozen medium were thawed and assayed by the COATEST VIII:C/4 kit (Chromogenix) per manufacturer's instructions.

A total of 10 individual BDD FVIII mutants were expressed. Four or two mutants were expressed at a time and on each occasion wild-type BDD FVIII was expressed in parallel in order to compare the activity of each mutant with that of wild-type BDD FVIII. Results obtained with wild-type BDD FVIII and the 10 BDD FVIII mutants are shown in Table 5.

TABLE 5

| | FVIII | CoA | Specific | Relative |
|---|---|---|---|---|
| | ELISA | activity | CoA activity | specific |
| Molecule | ng/ml | mU/ml | mU/ng | CoA activity |
| F8-500 = wild-type | 114 | 1100 | 9.6 | 1.0 |
| F8-500-L486N | 138 | 650 | 4.7 | 0.5 |
| F8-500-S488C | 93 | 725 | 7.8 | 0.8 |
| F8-500-K496C | 97 | 915 | 9.4 | 1.0 |
| F8-500-K496N-L498S | 150 | 1205 | 8.0 | 0.8 |
| F8-500 = wild-type | 52 | 610 | 11.7 | 1.0 |
| F8-500-T435C | 34 | 417 | 12.3 | 1.1 |
| F8-500-T435N-K437T | 66 | 720 | 10.9 | 0.9 |
| F8-500-S488N-R490T | 133 | 1207 | 9.1 | 0.8 |
| F8-500-L504C | 36 | 453 | 12.6 | 1.1 |
| F8-500 = wild-type | 37 | 517 | 14.0 | 1.0 |
| F8-500-D433N | 34 | 436 | 12.8 | 0.9 |
| F8-500-K377C | 21 | 197 | 9.4 | 0.7 |

### Example 4

### Determination of FVIII Activities in Culture Media by an Endogenous Thrombin Potential (ETP) Bioassay

ETP Assay Principle:

The endogenous thrombin potential (ETP) assay is based on a real time determination of thrombin generation in a selected plasma sample. The plasma sample contains thrombocytes as a physiological surface source for tenase and prothrombinase complexes in the coagulation cascade.

The real time thrombin activity is determined by continuous detection of an appearing fluorescent product from a thrombin specific substrate (Hemker et al., 2000; Hemker et al., 1993; Hemker & Béguin, 1995).

Materials:

FVIII deficient plasma (George King Bio-Medical Cat. No. 800-255-5108)

Lyophilised human thrombocytes (Biopool/Trinity ref. 50710)

Innovin (Dade Behring Prod. No. B 4212-50)

Thrombin specific substrate, e.g. Z-Gly-Gly-Arg-AMC HCl Fluorophor (Bachem Prod. No. I-1140)

Factor VIII standard (E.g. ReFacto (Wyeth))

FVIII-mutant containing media

Assay buffer: Tris-HCl, 50 mM; NaCl, 150 mM; CaCl2, Glycerol, 10%; Tween 8, 0.02%

Dilution buffer: Hepes, 20 mM; NaCl, 150 mM; pH 7.35; 2% bovine albumin

Procedure:

Thrombocytes were reconstituted in 1 ml reconstitution buffer (TBS) and further diluted in FVIII deficient plasma.

Innovin and Factor VIII were diluted in dilution buffer and assay buffer, respectively.

Z-Gly-Gly-Arg-AMC was dissolved in DMSO and further diluted in dilution buffer with CaCl₂.

Innovin (10 μl, final 0.12 μM), Factor VIII (10 μl, final 0-1 U/ml) and thrombocyte containing plasma (80 μl, final 50.000 thrombocytes/μl) was added to respective wells of a Costar plate (96 wells, Prod. No. 3631).

Blank or mutant containing media (10 μl) was added to respective wells.

The plate was incubated for 10 min at 37° C. in Thermo Fluoroscan Ascent. Substrate (20 μl, final 16.7 mM CaCl₂, 0.5% DMSO, 0.4 mM Thrombin specific substrate was immediately added and fluorescence was continuously recorded for 60 minutes (excitation 355 nm, emission 460 nm).

A standard curve was generated for a representative of the Factor VIII signal (Time to 250 fluorescent units). From this standard curve the level of Factor VIII-like activity in culture media was determined (Table 6).

TABLE 6
ETP and CoA activities determined in media of selected
FVIII analogues

| | | CoA activity, | ETP activity, |
|---|---|---|---|
| Sample | Molecule | mU/ml | mU/ml |
| 828 | F8-500 = B domain deleted | 1100 | 1700 |
| | FVIII | | |
| 842 | F8-500-L486N | 650 | 70 |
| 844 | F8-500-S488C | 725 | 1400 |
| 845 | F8-500-K496C | 915 | 2400 |
| 848 | F8-500-K496N-L498S | 1205 | 2700 |
| K1 | Medium, non-transfected cells | — | — |
| K2 | Fresh medium | — | — |

### Example 5

### Construction of A2 Domain Mutants

First the alpha-1 antitrypsin signal peptide cDNA is made in the pBluescript II SK+ plasmid. Two complementary oligonucleotides, AATTCGCCACCATGCCCTCGAGCGTCTCGTGGGGCATCCTCCTGCTGGCAGGCCTGTG CTGCCTGGTCCCTGTCTCGCTAGCTCTGCA (SEQ ID NO:25) and GAGCTAGCGAGACAGGGACCAGGCAGCACAGGCCTGCCAGCAGGAGGATGCCCCACG AGACGCTCGAGGGCATGGTGGCG (SEQ ID NO:26) both with a 5′-phosphate were annealed as follows. 90 picomole of each oligonucleotide in 0.12 M Tris-HCl [pH=7.5], 12 mM MgCl₂ were incubated 65° C. for 5 minutes, 37° C. for 10 minutes and finally RT for 10 minutes. The annealed oligonucleotides were ligated into the pBluescript II SK+ plasmid (Stratagene, La Jolla, Calif.) digested with EcoRI and PstI restriction enzymes. The product of the ligation is the coding DNA of the alpha-1 antitrypsin signal peptide, called “A1AT in pBluescript”.

The coding DNA region of the Factor VIII A2 domain was PCR amplified using the primers CGCTAGCTAAAACTTGGGTACATTACATTGCTG (SEQ ID NO: 27) and AGCGGCCGCTCTAACAACTAGAAACCTTCAGTAAGG (SEQ ID NO:28) on F8-500 in pTT5 plasmid (SEQ ID NO: 2) template, the 1 kb PCR band was TOPO-blunt (Invitrogen) cloned and sequence verified (sequencing service at MWG Biotech AG, Germany). The FVIII A2 fragment was transferred from the TOPO-blunt vector into the “A1AT in pBluescript” plasmid using the NheI and NotI restriction enzymes, forming the A1AT-FVIII-A2 construction. The “A1AT-FVIII-A2” fragment was then transferred from the pBluescript II SK+ plasmid into pTT5 using the restriction sites EcoRI and NotI, the resulting vector is called A1AT-FVIII-A2 in pTT5.

Next, a DNA fragment encoding the HPC4-tag (edqvdprlidgk) was inserted to the “A1AT-FVIII-A2 in pTT5” plasmid. The most N-terminal part of FVIII-A2 was amplified by PCR using the primers GGAGTGCAGCTTGAGGATCCAGAG (SEQ ID NO:29) and AGCGGCCGCTCTATTTGCCATCAATCAGGCGCGGATCCACCTGATCTTCGCCGGAGAA GCTTCTTGGTTCAATGG (SEQ ID NO:30) and the “F8-500 in pTT5” as template. The reverse primer harbour DNA sequences encoding the HPC4-tag. A PCR-product of 452 bp was transferred into the “A1AT-FVIII-A2 in pTT5”-construction using the restriction enzymes BsgI and NotI. The product is called A1AT-FVIII-A2-HPC4.

The mutations described in example 1 were then transferred from the “F8-500 in pTT5”-constructions to “A1AT-FVIII-A2-HPC4 in pTT5” using the PsiI and KpnI restriction enzymes.

### Example 6

### Transient Expression of A2 Domain Mutants

Suspension adapted human embryonal kidney (HEK293F) cells (Freestyle, Invitrogen) were transfected per manufacturer's instructions with expression plasmids encoding the HPC4-tagged A2 domain of wild-type FVIII or HPC4-tagged A2 domain with the above mutations (see ex. 5). Briefly, 30 μg of plasmid was incubated 20 min with 40 μl 293fectin (Invitrogen) and added to 3×10⁷ cells in a 125 ml Erlenmeyer flask. The transfected cells were incubated in a shaking incubator (37° C., 8% CO² and 125 rpm). Two days post-transfection, the cells were moved to a 27° C. shaking incubator. Four days post-transfection, the culture was centrifugated 1500×g for 5 min, and the cell pellet was discarded. The supernatant was frozen at −80° C. until purification of the tagged A2 domains.

### Purification of Tagged A2 Domain Mutants

Materials: 1 ml anti-protein C affinity matrix columns were from Roche Applied Science. Dialysis cassettes (Slide-A-Lyzer, MW cut-off: 3,500) were from Pierce. HBS-P buffer (10 mM HEPES, 150 mM NaCl, 0.005% P20) and 10% P20 was supplied from Biacore AB.

Buffer A: 10 mM HEPES, 150 mM, 1 mM CaCl2, 0.005% p20, pH 7.4. Buffer B: 10 mM HEPES, 1 M NaCl, 1 mM CaCl2, 0.005% P20, pH 7.4. Buffer C: 10 mM HEPES, 150 mM NaCl, 5 mM EDTA, 0.005% P20, pH 7.4.

Purification: The transiently expressed HPC4 tagged A2 mutant domains were purified by use of 1 ml anti-protein C affinity matrix columns. The 30 ml frozen (−80° C.) HEK293 freestyle transient transfection medium containing HPC4 tagged A2 domain was thawed and supplemented with 5 mM CaCl2 and 0.005% P20. The solution was filtered (22 μm) and loaded onto the anti-protein C column equilibrated in buffer A at room temperature. This step was followed by wash with 20 ml buffer B. Elution of the bound protein was obtained in buffer C. A flow rate of 0.5 ml/min was employed all through the experiment. The eluted protein was dialysed at 4° C. overnight using dialysis cassettes into HBS-P buffer supplemented with 5 mM CaCl2.

Concentration: Concentration determination of the purified A2 mutant domains were obtained using A280. Theoretical extinction coefficients and an Mw of 38,370 Da were obtained from Expasy proteomics server.

### Example 7

### Binding Analysis of A2 Domain Mutants to LRP

Materials: Degassed HBS-P buffer (10 mM HEPES, 150 mM NaCl, 0.005% P20), CM5 sensor chips, N-hydroxysuccinimide (NHS), ethyl-3(3-dimethyl-aminopropyl) carbodiimide (EDC) and ethanolamine were supplied by Biacore AB. Tagged A2 domain mutants were purified (described above) and delivered in the HBS-P buffer supplemented with 5 mM CaCl2. LRP was supplied freeze-dried from BioMac (Germany) and reconstituted in 20 mM HEPES, 150 mM NaCl, 5 mM CaCl2 and 0.05% Tween-20, pH 7.4 to a concentration of 0.5 mg/ml according to manufacturer. All other reagents were of analytical grade or better.

Immobilisation: Immobilisation of LRP was obtained by the standard procedure (supplied by manufacturer) for CM5 immobilisation using NHS and EDC and ethanolamine. Prior to immobilisation, LRP was diluted to a concentration of 25 μg/ml in 10 mM sodium acetate buffer, pH 3. The immobilisation was obtained at a density of 5-15 fmol/mm² (2000-6000 RU) in flow cell 2 of the CM5 sensor chip.

Binding analysis: Binding of A2 wt, A2 433D->N, A2 488S->N & 490R->T and A2 466K->A & 484R->A & 489R->A to immobilized soluble LRP were analyzed by surface plasmon resonance on a Biacore 3000 instrument. This approach is essentially described by Sarafanov et al., 2006.

The running buffer in use for the instrument was HBS-P supplemented with CaCl₂ to a final concentration of 5 mM. Kinetic analysis was performed at 25° C. at a flow rate of 30 μl/min running buffer. The untreated flow cell 1 was used for automatic in-line reference subtraction. Serial 2-fold dilutions of the A2 domains from 500 nM to 62.5 nM were analyzed. Following 3-min equilibration of the flow cells in running buffer, 150 μl protein samples were injected using the KINJECT command. The dissociation phase lasted 5 min and regeneration was performed with a 3-min pulse of 20 mM EDTA in HBS-P buffer. Surface plasmon resonance (SPR) data were fitted to 1:1 Langmuir binding model (supplied by the software) using BIAevaluation 4.1 software (Biacore AB). Results obtained with A2 wild type and three A2 domain mutants are presented in table 7.

TABLE 7
Binding constants (K_{D}) of A2 mutant domains to immobilised LRP.

| A2 domain mutant ID | K_{D} |
|---|---|
| A2 wt | 20 nM |
| A2 433D->N | 170 nM |
| A2 S488S->N & 490R->T | 260 nM |

### REFERENCES

Chau, M. H. and Nelson, J. W. (1991). Direct measurement of the equilibrium between glutathione and dithiothreitol by high performance liquid chromatography. FEBS Lett. 291, 296-298.
Grimberg, J., Maguire, S., and Belluscio, L. (1989). A simple method for the preparation of plasmid and chromosomal E. coli DNA. Nucleic Acids Res 17, 8893.
Riddles, P. W., Blakeley, R. L., and Zerner, B. (1979). Ellman's reagent: 5,5′-dithiobis(2-nitrobenzoic acid)—a reexamination. Anal. Biochem. 94, 75-81.
Sambrook, J., Fritsch, E. F., and Maniatis, T. (1989). Molecular Cloning: A Laboratory Manual. (Cold Spring Harbor, N.Y.: Cold Spring Harbor Laboratory).
Sarafanov A G, Makogonenko E M, Pechik I V, Ratke K P, Khrenov A V, Ananyeva N M, Strickland D K and Saenko E L. Identification of Coagulation Factor VIII A2 Domain Residues Forming the Binding Epitope for Low-Density Lipoprotein Receptor-Related Protein. Biochemistry. 2006, 45:1829-1840.
Vlamis-Gardikas, A., Aslund, F., Spyrou, G., Bergman, T., and Holmgren, A. (1997). Cloning, overexpression, and characterization of glutaredoxin 2, an atypical glutaredoxin from Escherichia coli. J. Biol. Chem. 272, 11236-11243.

1-42. (canceled)

43. A Factor VIII analog comprising amino acid substitution(s) of at least one of the amino acid residues corresponding to the amino acid residues in positions 430-520 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

44. The Factor VIII analog of claim 43, wherein at least one of the amino acid residues corresponding to (i) A430, I442, E445, I448, E456, V457, A469, Y476, T481, D482, R484, K499, T514, E518, and/or (ii) D433, E434, R439, S446, L452, G458, K466, S470, R471, V483, L486, R489, D500, F501, E507, I508, K512, and/or (iii) T432, T435, K437, T438, E440, A441, Q443, H444, Q468, Y487, S488, L491, G494, K496, H497, L498, L504, G506, V517 of human Factor VIII is substituted with a Cys residue.

45. The Factor VIII analog of claim 44, wherein at least one of the substituting cysteine residue(s) is conjugated to a water soluble polymer.

46. The Factor VIII analog of claim 44, wherein at least one of the residues corresponding to the residues in positions 435, 488, 496, or 504 of human Factor VIII is a Cys residue in the Factor VIII analog.

47. The Factor VIII analog of claim 43, wherein the amino acid substitution(s) in positions 430-520 consist of one, two, or three substitutions at position(s) that correspond to positions of the human Factor VIII sequence selected from 433, 435, 437, 486, 488, 490, and 496.

48. The Factor VIII analog of claim 47, wherein the residue(s) corresponding with the residues in position(s) 433, 486, or both 433 and 486 of human Factor VIII is/are Asn residue(s).

49. The Factor VIII analog of claim 47, wherein the residue in the position corresponding to position 435 of human Factor VIII is an Asn residue and the residue in the position corresponding to position 437 of human Factor VIII is a Thr residue or Ser residue.

50. The Factor VIII analog of claim 47, wherein the residue in the position corresponding to position 488 of human Factor VIII is an Asn residue and the residue in the position corresponding to position 490 of human Factor VIII is a Thr residue or Ser residue.

51. The Factor VIII analog of claim 47, wherein the residue in the position corresponding to position 496 of human Factor VIII is an Asn residue and the residue in the position corresponding to position 498 of human Factor VIII is a Thr residue or Ser residue.

52. The Factor VIII analog of claim 44, wherein (a) the residue in the position corresponding to position 435 of human Factor VIII is an Asn residue and the residue in the position corresponding to position 437 of human Factor VIII is a Thr residue or Ser residue and/or (b) the residue in the position corresponding to position 488 of human Factor VIII is an Asn residue and the residue in the position corresponding to position 490 of human Factor VIII is a Thr residue or Ser residue.

53. The Factor VIII analog of claim 44, wherein (a) the residue(s) corresponding with the residues in position(s) 433, 486, or both 433 and 486 of human Factor VIII is/are Asn residue(s); and/or (b) the residue in the position corresponding to position 496 of human Factor VIII is an Asn residue and the residue in the position corresponding to position 498 of human Factor VIII is a Thr residue or Ser residue.

54. The Factor VIII analog of claim 47, wherein the one, two, or three amino acid substitution(s) comprise one or more substitutions selected from the group consisting of S488C, K496C, L498S, T435C, T435N, K437T, S488N, R490T, L504C, L486N, and D433N.

55. The Factor VIII analog of claim 43, wherein at least one N-glycosylation site is introduced into the Factor VIII analog (as compared to human Factor VIII) at a position starting at a residue that corresponds to a residue situation in a position of human Factor VIII selected from (i) A430, I442, E445, I448, E456, V457, A469, T481, D482, K499, T514, E518; and/or (ii) D433, E434, R439, S446, L452, G458, K466, L486, R489, E507, I508, K512; and/or (iii) T432, T435, K437, T438, E440, A441, Q443, H444, Q468, Y487, S488, G494, K496, H497, L498, G506, and V517.

56. The Factor VIII analog of claim 44, wherein at least one N-glycosylation site is introduced into the Factor VIII analog (as compared to human Factor VIII) at a position starting at a residue that corresponds to a residue situation in a position of human Factor VIII selected from (i) A430, I442, E445, I448, E456, V457, A469, T481, D482, K499, T514, E518; and/or (ii) D433, E434, R439, S446, L452, G458, K466, L486, R489, E507, I508, K512; and/or (iii) T432, T435, K437, T438, E440, A441, Q443, H444, Q468, Y487, S488, G494, K496, H497, L498, G506, and V517.

57. The Factor VIII analog of claim 43, wherein the Factor VIII analog lacks one or more parts or all of the B-domain of human Factor VIII.

58. The Factor VIII of claim 43, wherein the Factor VIII analog lacks at least the portion of human Factor VIII B-domain corresponding to the region defined by S750 to C1636 of human Factor VIII.

59. The Factor VIII of claim 44, wherein the Factor VIII analog lacks at least the portion of human Factor VIII B-domain corresponding to the region defined by S750 to C1636 of human Factor VIII.

60. The Factor VIII of claim 55, wherein the Factor VIII analog lacks at least the portion of human Factor VIII B-domain corresponding to the region defined by S750 to C1636 of human Factor VIII.

61. The Factor VIII analog according to claim 43, wherein the Factor VIII analog lacks at least the portion of human Factor VIII B-domain corresponding to the region defined by T760 to N1639 of human Factor VIII.

62. The Factor VIII analog according to claim 44, wherein the Factor VIII analog lacks at least the portion of human Factor VIII B-domain corresponding to the region defined by T760 to N1639 of human Factor VIII.

63. The Factor VIII analog according to claim 55, wherein the Factor VIII analog lacks at least the portion of human Factor VIII B-domain corresponding to the region defined by T760 to N1639 of human Factor VIII.

64. A pharmaceutical composition comprising a therapeutically effective amount of a Factor VIII analog according to claim 43 and a pharmaceutically acceptable carrier.

65. A pharmaceutical composition comprising a therapeutically effective amount of a Factor VIII analog according to claim 44 and a pharmaceutically acceptable carrier.

66. A pharmaceutical composition comprising a therapeutically effective amount of a Factor VIII analog according to claim 55 and a pharmaceutically acceptable carrier.

67. A pharmaceutical composition comprising a therapeutically effective amount of a Factor VIII analog according to claim 57 and a pharmaceutically acceptable carrier.

68. A method of treating a hemophilia patient comprising delivering to the patient a therapeutically effective amount of a Factor VIII analog comprising amino acid substitution(s) of at least one of the amino acid residues corresponding to the amino acid residues in positions 430-520 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

69. The method of claim 68, wherein at least one of the amino acid residues corresponding to (i) A430, I442, E445, I448, E456, V457, A469, Y476, T481, D482, R484, K499, T514, E518, and/or (ii) D433, E434, R439, S446, L452, G458, K466, S470, R471, V483, L486, R489, D500, F501, E507, I508, K512, and/or (iii) T432, T435, K437, T438, E440, A441, Q443, H444, Q468, Y487, S488, L491, G494, K496, H497, L498, L504, G506, V517 of human Factor VIII is substituted with a Cys residue in the Factor VIII analog.

70. The method of claim 69, wherein at least one of the substituting cysteine residue(s) is conjugated to a water soluble polymer.

71. The method of claim 69, wherein at least one of the residues corresponding to the residues in positions 435, 488, 496, or 504 of human Factor VIII is a Cys residue in the Factor VIII analog.

72. The method of claim 68, wherein the amino acid substitution(s) in positions 430-520 consist of one, two, or three substitutions at position(s) that correspond to positions of the human Factor VIII sequence selected from 433, 435, 437, 486, 488, 490, and 496 in the Factor VIII analog.

73. The method of claim 72, wherein the residue(s) corresponding with the residues in position(s) 433, 486, or both 433 and 486 of human Factor VIII is/are Asn residue(s) in the Factor VIII analog.

74. The method of claim 72, wherein the residue in the position corresponding to position 435 of human Factor VIII is an Asn residue and the residue in the position corresponding to position 437 of human Factor VIII is a Thr residue or Ser residue in the Factor VIII analog.

75. The method of claim 72, wherein the residue in the position corresponding to position 488 of human Factor VIII is an Asn residue and the residue in the position corresponding to position 490 of human Factor VIII is a Thr residue or Ser residue.

76. The method of claim 72, wherein the residue in the position corresponding to position 496 of human Factor VIII is an Asn residue and the residue in the position corresponding to position 498 of human Factor VIII is a Thr residue or Ser residue in the Factor VIII analog.

77. The method of claim 69, wherein (a) the residue in the position corresponding to position 435 of human Factor VIII is an Asn residue and the residue in the position corresponding to position 437 of human Factor VIII is a Thr residue or Ser residue; and/or (b) the residue in the position corresponding to position 488 of human Factor VIII is an Asn residue and the residue in the position corresponding to position 490 of human Factor VIII is a Thr residue or Ser residue in the Factor VIII analog; and/or (c) the residue(s) corresponding with the residues in position(s) 433, 486, or both 433 and 486 of human Factor VIII is/are Asn residue(s); and/or (d) the residue in the position corresponding to position 496 of human Factor VIII is an Asn residue and the residue in the position corresponding to position 498 of human Factor VIII is a Thr residue or Ser residue in the Factor VIII analog.

78. The method of claim 72, wherein the one, two, or three amino acid substitution(s) comprise one or more substitutions selected from the group consisting of S488C, K496C, L498S, T435C, T435N, K437T, S488N, R490T, L504C, L486N, and D433N in the Factor VIII analog.

79. The method of claim 68, wherein at least one N-glycosylation site is introduced into the Factor VIII analog (as compared to human Factor VIII) at a position starting at a residue that corresponds to a residue situation in a position of human Factor VIII selected from (i) A430, I442, E445, I448, E456, V457, A469, T481, D482, K499, T514, E518; and/or (ii) D433, E434, R439, S446, L452, G458, K466, L486, R489, E507, I508, K512; and/or (iii) T432, T435, K437, T438, E440, A441, Q443, H444, Q468, Y487, S488, G494, K496, H497, L498, G506, and V517 in the Factor VIII analog.

80. The method of claim 69, wherein at least one N-glycosylation site is introduced into the Factor VIII analog (as compared to human Factor VIII) at a position starting at a residue that corresponds to a residue situation in a position of human Factor VIII selected from (i) A430, I442, E445, I448, E456, V457, A469, T481, D482, K499, T514, E518; and/or (ii) D433, E434, R439, S446, L452, G458, K466, L486, R489, E507, I508, K512; and/or (iii) T432, T435, K437, T438, E440, A441, Q443, H444, Q468, Y487, S488, G494, K496, H497, L498, G506, and V517 in the Factor VIII analog.

81. The method of claim 68, wherein the Factor VIII analog lacks one or more parts or all of the B-domain of human Factor VIII.

82. The method of claim 81, wherein the Factor VIII analog lacks at least the portion of human Factor VIII B-domain corresponding to the region defined by (a) S750 to C1636 of human Factor VIII or (b) T760 to N1639 of human Factor VIII.

83. The method of claim 69, wherein the Factor VIII analog lacks at least the portion of human Factor VIII B-domain corresponding to a region defined by (a) S750 to C1636 of human Factor VIII or (b) T760 to N1639 of human Factor VIII.

84. The method of claim 79, wherein the Factor VIII analog lacks at least the portion of human Factor VIII B-domain corresponding to the region defined by (a) S750 to C1636 of human Factor VIII or (b) T760 to N1639 of human Factor VIII.

85. The method of claim 68, wherein the Factor VIII analog is delivered to the patient by administering a pharmaceutical formulation comprising the Factor VIII analog and a pharmaceutically acceptable carrier to the patient.

86. The method of claim 69, wherein the Factor VIII analog is delivered to the patient by administering a pharmaceutical formulation comprising the Factor VIII analog and a pharmaceutically acceptable carrier to the patient.

87. The method of claim 86, wherein the method comprises once a week administration of the pharmaceutical formulation.

88. The method of claim 70, wherein the Factor VIII analog is delivered to the patient by administering a pharmaceutical formulation comprising the Factor VIII analog and a pharmaceutically acceptable carrier to the patient once a week.

89. The method of claim 79, wherein the Factor VIII analog is delivered to the patient by administering a pharmaceutical formulation comprising the Factor VIII analog and a pharmaceutically acceptable carrier to the patient.

90. The method of claim 80, wherein the Factor VIII analog is delivered to the patient by administering a pharmaceutical formulation comprising the Factor VIII analog and a pharmaceutically acceptable carrier to the patient, at least one of the substituting cysteine residue(s) is conjugated to a water soluble polymer, and the method comprises once a week administration of the pharmaceutical formulation.

91. The method of claim 81, wherein the Factor VIII analog is delivered to the patient by administering a pharmaceutical formulation comprising the Factor VIII analog and a pharmaceutically acceptable carrier to the patient.

92. The method of claim 83, wherein the Factor VIII analog is delivered to the patient by administering a pharmaceutical formulation comprising the Factor VIII analog and a pharmaceutically acceptable carrier to the patient, at least one of the substituting cysteine residue(s) is conjugated to a water soluble polymer, and the method comprises once a week administration of the pharmaceutical formulation.

93. A Factor VIII analog comprising amino acid substitution(s) of at least one of the amino acid residues corresponding to the amino acid residues in positions 333-395 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

94. The Factor VIII analog of claim 93, wherein at least one of the amino acid residues corresponding to (i) W382, H384, Y385, E389, W393, and/or (ii) Q334, K376, H378, T381, V383, E390, E391, D392, D394, and/or (iii) R336, K377, K380 of human Factor VIII is substituted with a Cys residue.

95. The Factor VIII analog of claim 94, wherein the Factor VIII analog comprises the substitution K377C.

96. The Factor VIII analog of claim 94, wherein at least one of the substituting Cys residue(s) is conjugated to a water soluble polymer.

97. The Factor VIII analog of claim 96, wherein the water soluble polymer comprises a PEG.

98. The Factor VIII analog of claim 96, wherein the PEG has an average molecular weight of 2-40 kDa.

99. The Factor VIII analog of claim 95, wherein the substituted Cys residue is conjugated to a water soluble polymer.

100. The Factor VIII analog of claim 90, wherein the water soluble polymer comprises a PEG.

101. The Factor VIII analog of claim 100, wherein the PEG has an average molecular weight of 2-40 kDa.

102. The Factor VIII analog of claim 93, wherein at least one N-glycosylation site is introduced into the Factor VIII analog (as compared to human Factor VIII) at a position starting at a residue that corresponds to a residue situation in a position of human Factor VIII selected from (i) W382, H384, Y385, E389, W393; and/or (ii) Q334, K376, T381, V383, E390, E391; and/or (iii) R336 or K380 of the human FVIII molecule.

103. The Factor VIII analog of claim 94, wherein at least one N-glycosylation site is introduced into the Factor VIII analog (as compared to human Factor VIII) at a position starting at a residue that corresponds to a residue situation in a position of human Factor VIII selected from (i) W382, H384, Y385, E389, W393; and/or (ii) Q334, K376, T381, V383, E390, E391; and/or (iii) R336 or K380 of the human FVIII molecule.

104. The Factor VIII analog of claim 93, wherein the Factor VIII analog lacks one or more parts or all of the B-domain of human Factor VIII.

105. The Factor VIII analog of claim 104, wherein the Factor VIII analog lacks from about 75% to about 85% or from about 85% to about 95% of the B-domain of human Factor VIII.

106. The Factor VIII of claim 104, wherein the Factor VIII analog lacks at least the portion of human Factor VIII B-domain corresponding to the region defined by (a) S750 to C1636 of human Factor VIII or (b) T760 to N1639 of human Factor VIII.

107. The Factor VIII analog of claim 94, wherein the Factor VIII analog lacks one or more parts or all of the B-domain of human Factor VIII.

108. The Factor VIII analog of claim 107, wherein the Factor VIII analog lacks from about 75% to about 85% or from about 85% to about 95% of the B-domain of human Factor VIII.

109. The Factor VIII of claim 107, wherein the Factor VIII analog lacks at least the portion of human Factor VIII B-domain corresponding to the region defined by (a) S750 to C1636 of human Factor VIII or (b) T760 to N1639 of human Factor VIII.

110. The Factor VIII analog of claim 102, wherein the Factor VIII analog lacks one or more parts or all of the B-domain of human Factor VIII.

111. The Factor VIII analog of claim 110, wherein the Factor VIII analog lacks from about 75% to about 85% or from about 85% to about 95% of the B-domain of human Factor VIII.

112. The Factor VIII of claim 110, wherein the Factor VIII analog lacks at least the portion of human Factor VIII B-domain corresponding to the region defined by (a) S750 to C1636 or (b) T760 to N1639 of human Factor VIII.

113. A pharmaceutical composition comprising a therapeutically effective amount of a Factor VIII analog according to claim 93 and a pharmaceutically acceptable carrier.

114. A pharmaceutical composition comprising a therapeutically effective amount of a Factor VIII analog according to claim 95 and a pharmaceutically acceptable carrier.

115. A pharmaceutical composition comprising a therapeutically effective amount of a Factor VIII analog according to claim 98 and a pharmaceutically acceptable carrier.

116. A pharmaceutical composition comprising a therapeutically effective amount of a Factor VIII analog according to claim 102 and a pharmaceutically acceptable carrier.

117. A pharmaceutical composition comprising a therapeutically effective amount of a Factor VIII analog according to claim 107 and a pharmaceutically acceptable carrier.

118. A method of treating a hemophilia patient comprising delivering to the patient a therapeutically effective amount of a Factor VIII analog comprising amino acid substitution(s) of at least one of the amino acid residues corresponding to the amino acid residues in positions 333-395 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

119. The method of claim 118, wherein at least one of the amino acid residues corresponding to (i) W382, H384, Y385, E389, W393, and/or (ii) Q334, K376, H378, T381, V383, E390, E391, D392, D394, and/or (iii) R336, K377, K380 of human Factor VIII is substituted with a Cys residue in the Factor VIII analog.

120. The method of claim 119, wherein the Factor VIII analog comprises the substitution K377C.

121. The method of claim 119, wherein at least one of the substituting Cys residue(s) is conjugated to a water soluble polymer.

122. The method of claim 121, wherein the water soluble polymer comprises a PEG.

123. The method of claim 122, wherein the PEG has an average molecular weight of 2-40 kDa.

124. The method of claim 120, wherein the substituted Cys residue is conjugated to a water soluble polymer.

125. The method of claim 124, wherein the water soluble polymer comprises a PEG.

126. The method of claim 125, wherein the PEG has an average molecular weight of 2-40 kDa.

127. The method of claim 118, wherein at least one N-glycosylation site is introduced into the Factor VIII analog (as compared to human Factor VIII) at a position starting at a residue that corresponds to a residue situation in a position of human Factor VIII selected from (i) W382, H384, Y385, E389, W393; and/or (ii) Q334, K376, T381, V383, E390, E391; and/or (iii) R336 or K380 of the human FVIII molecule.

128. The method of claim 119, wherein at least one N-glycosylation site is introduced into the Factor VIII analog (as compared to human Factor VIII) at a position starting at a residue that corresponds to a residue situation in a position of human Factor VIII selected from (i) W382, H384, Y385, E389, W393; and/or (ii) Q334, K376, T381, V383, E390, E391; and/or (iii) R336 or K380 of the human FVIII molecule.

129. The method of claim 118, wherein the Factor VIII analog lacks one or more parts or all of the B-domain of human Factor VIII.

130. The method of claim 129, wherein the Factor VIII analog lacks from about 75% to about 85% or from about 85% to about 95% of the B-domain of human Factor VIII.

131. The method of claim 129, wherein the Factor VIII analog lacks at least the portion of human Factor VIII B-domain corresponding to the region defined by (a) S750 to C1636 of human Factor VIII or (b) T760 to N1639 of human Factor VIII.

132. The method of claim 119, wherein the Factor VIII analog lacks one or more parts or all of the B-domain of human Factor VIII.

133. The method of claim 132, wherein the Factor VIII analog lacks from about 75% to about 85% or from about 85% to about 95% of the B-domain of human Factor VIII.

134. The method of claim 132, wherein the Factor VIII analog lacks at least the portion of human Factor VIII B-domain corresponding to the region defined by (a) S750 to C1636 of human Factor VIII or (b) T760 to N1639 of human Factor VIII.

135. The method of claim 127, wherein the Factor VIII analog lacks one or more parts or all of the B-domain of human Factor VIII.

136. The method of claim 135, wherein the Factor VIII analog lacks from about 75% to about 85% or from about 85% to about 95% of the B-domain of human Factor VIII.

137. The method of claim 135, wherein the Factor VIII analog lacks at least the portion of human Factor VIII B-domain corresponding to the region defined by (a) S750 to C1636 or (b) T760 to N1639 of human Factor VIII.

138. The method of claim 118, wherein the Factor VIII analog is delivered to the patient by administering a pharmaceutical formulation comprising the Factor VIII analog and a pharmaceutically acceptable carrier to the patient.

139. The method of claim 121, wherein the Factor VIII analog is delivered to the patient by administering a pharmaceutical formulation comprising the Factor VIII analog and a pharmaceutically acceptable carrier to the patient once a week.

140. The method of claim 120, wherein the Factor VIII analog is delivered to the patient by administering a pharmaceutical formulation comprising the Factor VIII analog and a pharmaceutically acceptable carrier to the patient.

141. The method of claim 127, wherein the Factor VIII analog is delivered to the patient by administering a pharmaceutical formulation comprising the Factor VIII analog and a pharmaceutically acceptable carrier to the patient.

142. The method of claim 129, wherein the Factor VIII analog is delivered to the patient by administering a pharmaceutical formulation comprising the Factor VIII analog and a pharmaceutically acceptable carrier to the patient.

143. A Factor VIII analog comprising amino acid substitution(s) of at least one of the amino acid residues corresponding to the amino acid residues in positions 20-29 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

144. The Factor VIII analog of claim 143, wherein at least one of the amino acid residues corresponding to (i) L24 or D27 and/or (ii) D20, L21, E23, A28, or R29 of human Factor VIII is substituted with a Cys residue.

145. The Factor VIII analog of claim 144, wherein at least one of the substituting Cys residue(s) is conjugated to a water soluble polymer.

146. The Factor VIII analog of claim 145, wherein the water soluble polymer comprises a PEG.

147. The Factor VIII analog of claim 146, wherein the PEG has an average molecular weight of 2-40 kDa.

148. The Factor VIII analog of claim 143, wherein at least one N-glycosylation site is introduced into the Factor VIII analog (as compared to human Factor VIII) at a position starting at a residue that corresponds to a residue situation in a position of human Factor VIII selected from (i) D27; and/or (ii) D20, L21, or A28 of the human FVIII molecule.

149. The Factor VIII analog of claim 144, wherein at least one N-glycosylation site is introduced into the Factor VIII analog (as compared to human Factor VIII) at a position starting at a residue that corresponds to a residue situation in a position of human Factor VIII selected from (i) D27; and/or (ii) D20, L21, or A28 of the human FVIII molecule.

150. The Factor VIII analog of claim 143, wherein the Factor VIII analog lacks one or more parts or all of the B-domain of human Factor VIII.

151. The Factor VIII analog of claim 150, wherein the Factor VIII analog lacks from about 75% to about 85% or from about 85% to about 95% of the B-domain of human Factor VIII.

152. The Factor VIII of claim 150, wherein the Factor VIII analog lacks at least the portion of human Factor VIII B-domain corresponding to the region defined by (a) S750 to C1636 of human Factor VIII or (b) T760 to N1639 of human Factor VIII.

153. The Factor VIII analog of claim 144, wherein the Factor VIII analog lacks one or more parts or all of the B-domain of human Factor VIII.

154. The Factor VIII analog of claim 153, wherein the Factor VIII analog lacks from about 75% to about 85% or from about 85% to about 95% of the B-domain of human Factor VIII.

155. The Factor VIII of claim 153, wherein the Factor VIII analog lacks at least the portion of human Factor VIII B-domain corresponding to the region defined by (a) S750 to C1636 of human Factor VIII or (b) T760 to N1639 of human Factor VIII.

156. The Factor VIII analog of claim 148, wherein the Factor VIII analog lacks one or more parts or all of the B-domain of human Factor VIII.

157. The Factor VIII analog of claim 156, wherein the Factor VIII analog lacks from about 75% to about 85% or from about 85% to about 95% of the B-domain of human Factor VIII.

158. The Factor VIII of claim 157, wherein the Factor VIII analog lacks at least the portion of human Factor VIII B-domain corresponding to the region defined by (a) S750 to C1636 or (b) T760 to N1639 of human Factor VIII.

159. A pharmaceutical composition comprising a therapeutically effective amount of a Factor VIII analog according to claim 143 and a pharmaceutically acceptable carrier.

160. A pharmaceutical composition comprising a therapeutically effective amount of a Factor VIII analog according to claim 144 and a pharmaceutically acceptable carrier.

161. A pharmaceutical composition comprising a therapeutically effective amount of a Factor VIII analog according to claim 145 and a pharmaceutically acceptable carrier.

162. A pharmaceutical composition comprising a therapeutically effective amount of a Factor VIII analog according to claim 147 and a pharmaceutically acceptable carrier.

163. A pharmaceutical composition comprising a therapeutically effective amount of a Factor VIII analog according to claim 148 and a pharmaceutically acceptable carrier.

164. A pharmaceutical composition comprising a therapeutically effective amount of a Factor VIII analog according to claim 150 and a pharmaceutically acceptable carrier.

165. A method of treating a hemophilia patient comprising delivering to the patient a therapeutically effective amount of a Factor VIII analog comprising amino acid substitution(s) of at least one of the amino acid residues corresponding to the amino acid residues in positions 29-29 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

166. The method of claim 165, wherein at least one of the amino acid residues corresponding to (i) L24 or D27 and/or (ii) D20, L21, E23, A28, or R29 of human Factor VIII is substituted with a Cys residue in the Factor VIII analog.

167. The method of claim 166, wherein at least one of the substituting Cys residue(s) is conjugated to a water soluble polymer.

168. The method of claim 167, wherein the water soluble polymer comprises a PEG.

169. The method of claim 168, wherein the PEG has an average molecular weight of 2-40 kDa.

170. The method of claim 165, wherein at least one N-glycosylation site is introduced into the Factor VIII analog (as compared to human Factor VIII) at a position starting at a residue that corresponds to a residue situation in a position of human Factor VIII selected from (i) D27; and/or (ii) D20, L21, or A28 of the human FVIII molecule.

171. The method of claim 166, wherein at least one N-glycosylation site is introduced into the Factor VIII analog (as compared to human Factor VIII) at a position starting at a residue that corresponds to a residue situation in a position of human Factor VIII selected from (i) D27; and/or (ii) D20, L21, or A28 of the human FVIII molecule.

172. The method of claim 165, wherein the Factor VIII analog lacks one or more parts or all of the B-domain of human Factor VIII.

173. The method of claim 172, wherein the Factor VIII analog lacks from about 75% to about 85% or from about 85% to about 95% of the B-domain of human Factor VIII.

174. The method of claim 172, wherein the Factor VIII analog lacks at least the portion of human Factor VIII B-domain corresponding to the region defined by (a) S750 to C1636 of human Factor VIII or (b) T760 to N1639 of human Factor VIII.

175. The method of claim 166, wherein the Factor VIII analog lacks one or more parts or all of the B-domain of human Factor VIII.

176. The method of claim 175, wherein the Factor VIII analog lacks from about 75% to about 85% or from about 85% to about 95% of the B-domain of human Factor VIII.

177. The method of claim 175, wherein the Factor VIII analog lacks at least the portion of human Factor VIII B-domain corresponding to the region defined by (a) S750 to C1636 of human Factor VIII or (b) T760 to N1639 of human Factor VIII.

178. The method of claim 170, wherein the Factor VIII analog lacks one or more parts or all of the B-domain of human Factor VIII.

179. The method of claim 178, wherein the Factor VIII analog lacks from about 75% to about 85% or from about 85% to about 95% of the B-domain of human Factor VIII.

180. The method of claim 178, wherein the Factor VIII analog lacks at least the portion of human Factor VIII B-domain corresponding to the region defined by (a) S750 to C1636 or (b) T760 to N1639 of human Factor VIII.

181. The method of claim 165, wherein the Factor VIII analog is delivered to the patient by administering a pharmaceutical formulation comprising the Factor VIII analog and a pharmaceutically acceptable carrier to the patient.

182. The method of claim 167, wherein the Factor VIII analog is delivered to the patient by administering a pharmaceutical formulation comprising the Factor VIII analog and a pharmaceutically acceptable carrier to the patient.

183. The method of claim 182, wherein the method comprises once a week administration of the pharmaceutical formulation.

184. The method of claim 169, wherein the Factor VIII analog is delivered to the patient by administering a pharmaceutical formulation comprising the Factor VIII analog and a pharmaceutically acceptable carrier to the patient.

185. The method of claim 184, wherein the method comprises once a week administration of the pharmaceutical formulation.

186. The method of claim 171, wherein the Factor VIII analog is delivered to the patient by administering a pharmaceutical formulation comprising the Factor VIII analog and a pharmaceutically acceptable carrier to the patient.

187. The method of claim 172, wherein the Factor VIII analog is delivered to the patient by administering a pharmaceutical formulation comprising the Factor VIII analog and a pharmaceutically acceptable carrier to the patient.

188. The method of claim 176, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

189. The method of claim 188, wherein the water-soluble polymer is a PEG having an average molecular weight of 2-40 kDa and the method comprises once a week administration of the pharmaceutical formulation.

190. A Factor VIII analog comprising amino acid substitution(s) of at least one of the amino acid residues corresponding to the amino acid residues in positions 268-276 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

191. The Factor VIII analog of claim 190, wherein F276 of human Factor VIII is substituted with a Cys residue.

192. The Factor VIII analog of claim 191, wherein the substituting Cys residue is conjugated to a water soluble polymer.

193. The Factor VIII analog of claim 192, wherein the water soluble polymer comprises a PEG.

194. The Factor VIII analog of claim 193, wherein the PEG has an average molecular weight of 2-40 kDa.

195. The Factor VIII analog of claim 190, wherein at least one N-glycosylation site is introduced into the Factor VIII analog (as compared to human Factor VIII) at a position starting at a residue that corresponds to F276 of the human FVIII molecule.

196. The Factor VIII analog of claim 191, wherein the amino acid substitutions further introduce at least one N-glycosylation site into the Factor VIII analog (as compared to human Factor VIII).

197. The Factor VIII analog of claim 190, wherein the Factor VIII analog lacks one or more parts or all of the B-domain of human Factor VIII.

198. The Factor VIII analog of claim 197, wherein the Factor VIII analog lacks from about 75% to about 85% or from about 85% to about 95% of the B-domain of human Factor VIII.

199. The Factor VIII of claim 197, wherein the Factor VIII analog lacks at least the portion of human Factor VIII B-domain corresponding to the region defined by (a) S750 to C1636 of human Factor VIII or (b) T760 to N1639 of human Factor VIII.

200. The Factor VIII analog of claim 191, wherein the Factor VIII analog lacks one or more parts or all of the B-domain of human Factor VIII.

201. The Factor VIII analog of claim 200, wherein the Factor VIII analog lacks from about 75% to about 85% or from about 85% to about 95% of the B-domain of human Factor VIII.

202. The Factor VIII of claim 200, wherein the Factor VIII analog lacks at least the portion of human Factor VIII B-domain corresponding to the region defined by (a) S750 to C1636 of human Factor VIII or (b) T760 to N1639 of human Factor VIII.

203. The Factor VIII analog of claim 195, wherein the Factor VIII analog lacks one or more parts or all of the B-domain of human Factor VIII.

204. The Factor VIII analog of claim 203, wherein the Factor VIII analog lacks from about 75% to about 85% or from about 85% to about 95% of the B-domain of human Factor VIII.

205. The Factor VIII of claim 203, wherein the Factor VIII analog lacks at least the portion of human Factor VIII B-domain corresponding to the region defined by (a) S750 to C1636 or (b) T760 to N1639 of human Factor VIII.

206. A pharmaceutical composition comprising a therapeutically effective amount of a Factor VIII analog according to claim 190 and a pharmaceutically acceptable carrier.

207. A pharmaceutical composition comprising a therapeutically effective amount of a Factor VIII analog according to claim 191 and a pharmaceutically acceptable carrier.

208. A pharmaceutical composition comprising a therapeutically effective amount of a Factor VIII analog according to claim 192 and a pharmaceutically acceptable carrier.

209. A pharmaceutical composition comprising a therapeutically effective amount of a Factor VIII analog according to claim 194 and a pharmaceutically acceptable carrier.

210. A pharmaceutical composition comprising a therapeutically effective amount of a Factor VIII analog according to claim 195 and a pharmaceutically acceptable carrier.

211. A pharmaceutical composition comprising a therapeutically effective amount of a Factor VIII analog according to claim 197 and a pharmaceutically acceptable carrier.

212. A pharmaceutical composition comprising a therapeutically effective amount of a Factor VIII analog according to claim 198 and a pharmaceutically acceptable carrier.

213. A pharmaceutical composition comprising a therapeutically effective amount of a Factor VIII analog according to claim 199 and a pharmaceutically acceptable carrier.

214. A pharmaceutical composition comprising (a) a therapeutically effective amount of a Factor VIII analog according to claim 201, wherein the substituting Cys residue is conjugated to a PEG having an average molecular weight of 2-40 kDa, and (b) a pharmaceutically acceptable carrier.

215. A method of treating a hemophilia patient comprising delivering to the patient a therapeutically effective amount of a Factor VIII analog comprising amino acid substitution(s) of at least one of the amino acid residues corresponding to the amino acid residues in positions 268-276 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

216. The method of claim 215, wherein F276 of human Factor VIII is substituted with a Cys residue in the Factor VIII analog.

217. The method of claim 216, wherein the substituting Cys residue is conjugated to a water soluble polymer.

218. The method of claim 217, wherein the water soluble polymer comprises a PEG.

219. The method of claim 218, wherein the PEG has an average molecular weight of 2-40 kDa.

220. The method of claim 215, wherein at least one N-glycosylation site is introduced into the Factor VIII analog (as compared to human Factor VIII) at a position starting at a residue that corresponds to F276 of the human FVIII molecule.

221. The method of claim 216, wherein the amino acid substitutions further introduce at least one N-glycosylation site into the Factor VIII analog (as compared to human Factor VIII).

222. The method of claim 215, wherein the Factor VIII analog lacks one or more parts or all of the B-domain of human Factor VIII.

223. The method of claim 222, wherein the Factor VIII analog lacks from about 75% to about 85% or from about 85% to about 95% of the B-domain of human Factor VIII.

224. The method of claim 222, wherein the Factor VIII analog lacks at least the portion of human Factor VIII B-domain corresponding to the region defined by (a) S750 to C1636 of human Factor VIII or (b) T760 to N1639 of human Factor VIII.

225. The method of claim 216, wherein the Factor VIII analog lacks one or more parts or all of the B-domain of human Factor VIII.

226. The method of claim 225, wherein the Factor VIII analog lacks from about 75% to about 85% or from about 85% to about 95% of the B-domain of human Factor VIII.

227. The method of claim 225, wherein the Factor VIII analog lacks at least the portion of human Factor VIII B-domain corresponding to the region defined by (a) S750 to C1636 of human Factor VIII or (b) T760 to N1639 of human Factor VIII.

228. The method of claim 220, wherein the Factor VIII analog lacks one or more parts or all of the B-domain of human Factor VIII.

229. The method of claim 228, wherein the Factor VIII analog lacks from about 75% to about 85% or from about 85% to about 95% of the B-domain of human Factor VIII.

230. The method of claim 228, wherein the Factor VIII analog lacks at least the portion of human Factor VIII B-domain corresponding to the region defined by (a) S750 to C1636 or (b) T760 to N1639 of human Factor VIII.

231. The method of claim 215, wherein the Factor VIII analog is delivered to the patient by administering a pharmaceutical formulation comprising the Factor VIII analog and a pharmaceutically acceptable carrier to the patient.

232. The method of claim 217, wherein the Factor VIII analog is delivered to the patient by administering a pharmaceutical formulation comprising the Factor VIII analog and a pharmaceutically acceptable carrier to the patient.

233. The method of claim 232, wherein the method comprises once a week administration of the pharmaceutical formulation.

234. The method of claim 219, wherein the Factor VIII analog is delivered to the patient by administering a pharmaceutical formulation comprising the Factor VIII analog and a pharmaceutically acceptable carrier to the patient.

235. The method of claim 234, wherein the method comprises once a week administration of the pharmaceutical formulation.

236. The method of claim 220, wherein the Factor VIII analog is delivered to the patient by administering a pharmaceutical formulation comprising the Factor VIII analog and a pharmaceutically acceptable carrier to the patient.

237. The method of claim 222, wherein the Factor VIII analog is delivered to the patient by administering a pharmaceutical formulation comprising the Factor VIII analog and a pharmaceutically acceptable carrier to the patient.

238. The method of claim 226, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

239. The method of claim 238, wherein the water-soluble polymer is a PEG having an average molecular weight of 2-40 kDa and the method comprises once a week administration of the pharmaceutical formulation.

240. A Factor VIII analog comprising amino acid substitution(s) of at least one of the amino acid residues corresponding to the amino acid residues in positions 302-313 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

241. The Factor VIII analog of claim 240, wherein at least one of the amino acid residues corresponding to (i) F306 or L307 and/or (ii) L303, G304, or Q305 of human Factor VIII is substituted with a Cys residue.

242. The Factor VIII analog of claim 241, wherein at least one of the substituting Cys residue(s) is conjugated to a water soluble polymer.

243. The Factor VIII analog of claim 242, wherein the water soluble polymer comprises a PEG.

244. The Factor VIII analog of claim 243, wherein the PEG has an average molecular weight of 2-40 kDa.

245. The Factor VIII analog of claim 240, wherein at least one N-glycosylation site is introduced into the Factor VIII analog (as compared to human Factor VIII) at a position starting at a residue that corresponds to a residue situation in a position of human Factor VIII selected from (i) F306 or L307; and/or (ii) L303, G304, or Q305 of the human FVIII molecule.

246. The Factor VIII analog of claim 241, wherein at least one N-glycosylation site is introduced into the Factor VIII analog (as compared to human Factor VIII) at a position starting at a residue that corresponds to a residue situation in a position of human Factor VIII selected from (i) F306 or L307; and/or (ii) L303, G304, or Q305 of the human FVIII molecule.

247. The Factor VIII analog of claim 240, wherein the Factor VIII analog lacks one or more parts or all of the B-domain of human Factor VIII.

248. The Factor VIII analog of claim 247, wherein the Factor VIII analog lacks from about 75% to about 85% or from about 85% to about 95% of the B-domain of human Factor VIII.

249. The Factor VIII of claim 247, wherein the Factor VIII analog lacks at least the portion of human Factor VIII B-domain corresponding to the region defined by (a) S750 to C1636 of human Factor VIII or (b) T760 to N1639 of human Factor VIII.

250. The Factor VIII analog of claim 241, wherein the Factor VIII analog lacks one or more parts or all of the B-domain of human Factor VIII.

251. The Factor VIII analog of claim 250, wherein the Factor VIII analog lacks from about 75% to about 85% or from about 85% to about 95% of the B-domain of human Factor VIII.

252. The Factor VIII of claim 250, wherein the Factor VIII analog lacks at least the portion of human Factor VIII B-domain corresponding to the region defined by (a) S750 to C1636 of human Factor VIII or (b) T760 to N1639 of human Factor VIII.

253. The Factor VIII analog of claim 245, wherein the Factor VIII analog lacks one or more parts or all of the B-domain of human Factor VIII.

254. The Factor VIII analog of claim 253, wherein the Factor VIII analog lacks from about 75% to about 85% or from about 85% to about 95% of the B-domain of human Factor VIII.

255. The Factor VIII of claim 253, wherein the Factor VIII analog lacks at least the portion of human Factor VIII B-domain corresponding to the region defined by (a) S750 to C1636 or (b) T760 to N1639 of human Factor VIII.

256. A pharmaceutical composition comprising a therapeutically effective amount of a Factor VIII analog according to claim 240 and a pharmaceutically acceptable carrier.

257. A pharmaceutical composition comprising a therapeutically effective amount of a Factor VIII analog according to claim 241 and a pharmaceutically acceptable carrier.

258. A pharmaceutical composition comprising a therapeutically effective amount of a Factor VIII analog according to claim 242 and a pharmaceutically acceptable carrier.

259. A pharmaceutical composition comprising a therapeutically effective amount of a Factor VIII analog according to claim 244 and a pharmaceutically acceptable carrier.

260. A pharmaceutical composition comprising a therapeutically effective amount of a Factor VIII analog according to claim 245 and a pharmaceutically acceptable carrier.

261. A pharmaceutical composition comprising a therapeutically effective amount of a Factor VIII analog according to claim 247 and a pharmaceutically acceptable carrier.

262. A pharmaceutical composition comprising a therapeutically effective amount of a Factor VIII analog according to claim 248 and a pharmaceutically acceptable carrier.

263. A pharmaceutical composition comprising a therapeutically effective amount of a Factor VIII analog according to claim 249 and a pharmaceutically acceptable carrier.

264. A pharmaceutical composition comprising (a) a therapeutically effective amount of a Factor VIII analog according to claim 251, wherein the substituting Cys residue is conjugated to a PEG having an average molecular weight of 2-40 kDa, and (b) a pharmaceutically acceptable carrier.

265. A method of treating a hemophilia patient comprising delivering to the patient a therapeutically effective amount of a Factor VIII analog comprising amino acid substitution(s) of at least one of the amino acid residues corresponding to the amino acid residues in positions 302-313 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

266. The method of claim 265, wherein at least one of the amino acid residues corresponding to (i) F306 or L307 and/or (ii) L303, G304, or Q305 of human Factor VIII is substituted with a Cys residue in the Factor VIII analog.

267. The method of claim 266, wherein at least one of the substituting Cys residue(s) is conjugated to a water soluble polymer.

268. The method of claim 267, wherein the water soluble polymer comprises a PEG.

269. The method of claim 268, wherein the PEG has an average molecular weight of 2-40 kDa.

270. The method of claim 265, wherein at least one N-glycosylation site is introduced into the Factor VIII analog (as compared to human Factor VIII) at a position starting at a residue that corresponds to a residue situation in a position of human Factor VIII selected from (i) F306 or L307; and/or (ii) L303, G304, or Q305 of the human FVIII molecule.

271. The method of claim 266, wherein at least one N-glycosylation site is introduced into the Factor VIII analog (as compared to human Factor VIII) at a position starting at a residue that corresponds to a residue situation in a position of human Factor VIII selected from (i) F306 or L307; and/or (ii) L303, G304, or Q305 of the human FVIII molecule.

272. The method of claim 265, wherein the Factor VIII analog lacks one or more parts or all of the B-domain of human Factor VIII.

273. The method of claim 272, wherein the Factor VIII analog lacks from about 75% to about 85% or from about 85% to about 95% of the B-domain of human Factor VIII.

274. The method of claim 272, wherein the Factor VIII analog lacks at least the portion of human Factor VIII B-domain corresponding to the region defined by (a) S750 to C1636 of human Factor VIII or (b) T760 to N1639 of human Factor VIII.

275. The method of claim 266, wherein the Factor VIII analog lacks one or more parts or all of the B-domain of human Factor VIII.

276. The method of claim 275, wherein the Factor VIII analog lacks from about 75% to about 85% or from about 85% to about 95% of the B-domain of human Factor VIII.

277. The method of claim 275, wherein the Factor VIII analog lacks at least the portion of human Factor VIII B-domain corresponding to the region defined by (a) S750 to C1636 of human Factor VIII or (b) T760 to N1639 of human Factor VIII.

278. The method of claim 270, wherein the Factor VIII analog lacks one or more parts or all of the B-domain of human Factor VIII.

279. The method of claim 278, wherein the Factor VIII analog lacks from about 75% to about 85% or from about 85% to about 95% of the B-domain of human Factor VIII.

280. The method of claim 278, wherein the Factor VIII analog lacks at least the portion of human Factor VIII B-domain corresponding to the region defined by (a) S750 to C1636 or (b) T760 to N1639 of human Factor VIII.

281. The method of claim 265, wherein the Factor VIII analog is delivered to the patient by administering a pharmaceutical formulation comprising the Factor VIII analog and a pharmaceutically acceptable carrier to the patient.

282. The method of claim 267, wherein the Factor VIII analog is delivered to the patient by administering a pharmaceutical formulation comprising the Factor VIII analog and a pharmaceutically acceptable carrier to the patient.

283. The method of claim 282, wherein the method comprises once a week administration of the pharmaceutical formulation.

284. The method of claim 269, wherein the Factor VIII analog is delivered to the patient by administering a pharmaceutical formulation comprising the Factor VIII analog and a pharmaceutically acceptable carrier to the patient.

285. The method of claim 284, wherein the method comprises once a week administration of the pharmaceutical formulation.

286. The method of claim 270, wherein the Factor VIII analog is delivered to the patient by administering a pharmaceutical formulation comprising the Factor VIII analog and a pharmaceutically acceptable carrier to the patient.

287. The method of claim 272, wherein the Factor VIII analog is delivered to the patient by administering a pharmaceutical formulation comprising the Factor VIII analog and a pharmaceutically acceptable carrier to the patient.

288. The method of claim 276, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

289. The method of claim 288, wherein the water-soluble polymer is a PEG having an average molecular weight of 2-40 kDa and the method comprises once a week administration of the pharmaceutical formulation.

290. A Factor VIII analog comprising amino acid substitution(s) of at least one of the amino acid residues corresponding to the amino acid residues in positions 321-326 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

291. The Factor VIII analog of claim 290, wherein at least one of the amino acid residues corresponding to (i) Y323 and/or (ii) K325 of human Factor VIII is substituted with a Cys residue.

292. The Factor VIII analog of claim 291, wherein at least one of the substituting Cys residue(s) is conjugated to a water soluble polymer.

293. The Factor VIII analog of claim 292, wherein the water soluble polymer comprises a PEG.

294. The Factor VIII analog of claim 293, wherein the PEG has an average molecular weight of 2-40 kDa.

295. The Factor VIII analog of claim 290, wherein at least one N-glycosylation site is introduced into the Factor VIII analog (as compared to human Factor VIII) at a position starting at a residue that corresponds to a residue situation in a position of human Factor VIII selected from (i) Y323 and/or (ii) K325 of the human FVIII molecule.

296. The Factor VIII analog of claim 291, wherein at least one N-glycosylation site is introduced into the Factor VIII analog (as compared to human Factor VIII) at a position starting at a residue that corresponds to a residue situation in a position of human Factor VIII selected from (i) Y323 and/or (ii) K325 of the human FVIII molecule.

297. The Factor VIII analog of claim 290, wherein the Factor VIII analog lacks one or more parts or all of the B-domain of human Factor VIII.

298. The Factor VIII analog of claim 297, wherein the Factor VIII analog lacks from about 75% to about 85% or from about 85% to about 95% of the B-domain of human Factor VIII.

299. The Factor VIII of claim 297, wherein the Factor VIII analog lacks at least the portion of human Factor VIII B-domain corresponding to the region defined by (a) S750 to C1636 of human Factor VIII or (b) T760 to N1639 of human Factor VIII.

300. The Factor VIII analog of claim 291, wherein the Factor VIII analog lacks one or more parts or all of the B-domain of human Factor VIII.

301. The Factor VIII analog of claim 300, wherein the Factor VIII analog lacks from about 75% to about 85% or from about 85% to about 95% of the B-domain of human Factor VIII.

302. The Factor VIII of claim 300, wherein the Factor VIII analog lacks at least the portion of human Factor VIII B-domain corresponding to the region defined by (a) S750 to C1636 of human Factor VIII or (b) T760 to N1639 of human Factor VIII.

303. The Factor VIII analog of claim 295, wherein the Factor VIII analog lacks one or more parts or all of the B-domain of human Factor VIII.

304. The Factor VIII analog of claim 303, wherein the Factor VIII analog lacks from about 75% to about 85% or from about 85% to about 95% of the B-domain of human Factor VIII.

305. The Factor VIII of claim 303, wherein the Factor VIII analog lacks at least the portion of human Factor VIII B-domain corresponding to the region defined by (a) S750 to C1636 or (b) T760 to N1639 of human Factor VIII.

306. A pharmaceutical composition comprising a therapeutically effective amount of a Factor VIII analog according to claim 290 and a pharmaceutically acceptable carrier.

307. A pharmaceutical composition comprising a therapeutically effective amount of a Factor VIII analog according to claim 291 and a pharmaceutically acceptable carrier.

308. A pharmaceutical composition comprising a therapeutically effective amount of a Factor VIII analog according to claim 292 and a pharmaceutically acceptable carrier.

309. A pharmaceutical composition comprising a therapeutically effective amount of a Factor VIII analog according to claim 294 and a pharmaceutically acceptable carrier.

310. A pharmaceutical composition comprising a therapeutically effective amount of a Factor VIII analog according to claim 295 and a pharmaceutically acceptable carrier.

311. A pharmaceutical composition comprising a therapeutically effective amount of a Factor VIII analog according to claim 297 and a pharmaceutically acceptable carrier.

312. A pharmaceutical composition comprising a therapeutically effective amount of a Factor VIII analog according to claim 298 and a pharmaceutically acceptable carrier.

313. A pharmaceutical composition comprising a therapeutically effective amount of a Factor VIII analog according to claim 299 and a pharmaceutically acceptable carrier.

314. A pharmaceutical composition comprising (a) a therapeutically effective amount of a Factor VIII analog according to claim 301, wherein the substituting Cys residue is conjugated to a PEG having an average molecular weight of 2-40 kDa, and (b) a pharmaceutically acceptable carrier.

315. A method of treating a hemophilia patient comprising delivering to the patient a therapeutically effective amount of a Factor VIII analog comprising amino acid substitution(s) of at least one of the amino acid residues corresponding to the amino acid residues in positions 321-326 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

316. The method of claim 315, wherein at least one of the amino acid residues corresponding to (i) Y323 and/or (ii) K325 of human Factor VIII is substituted with a Cys residue in the Factor VIII analog.

317. The method of claim 316, wherein at least one of the substituting Cys residue(s) is conjugated to a water soluble polymer.

318. The method of claim 317, wherein the water soluble polymer comprises a PEG.

319. The method of claim 318, wherein the PEG has an average molecular weight of 2-40 kDa.

320. The method of claim 315, wherein at least one N-glycosylation site is introduced into the Factor VIII analog (as compared to human Factor VIII) at a position starting at a residue that corresponds to a residue situation in a position of human Factor VIII selected from (i) Y323 and/or (ii) K325 of the human FVIII molecule.

321. The method of claim 316, wherein at least one N-glycosylation site is introduced into the Factor VIII analog (as compared to human Factor VIII) at a position starting at a residue that corresponds to a residue situation in a position of human Factor VIII selected from (i) Y323 and/or (ii) K325 of the human FVIII molecule.

322. The method of claim 315, wherein the Factor VIII analog lacks one or more parts or all of the B-domain of human Factor VIII.

323. The method of claim 322, wherein the Factor VIII analog lacks from about 75% to about 85% or from about 85% to about 95% of the B-domain of human Factor VIII.

324. The method of claim 322, wherein the Factor VIII analog lacks at least the portion of human Factor VIII B-domain corresponding to the region defined by (a) S750 to C1636 of human Factor VIII or (b) T760 to N1639 of human Factor VIII.

325. The method of claim 316, wherein the Factor VIII analog lacks one or more parts or all of the B-domain of human Factor VIII.

326. The method of claim 325, wherein the Factor VIII analog lacks from about 75% to about 85% or from about 85% to about 95% of the B-domain of human Factor VIII.

327. The method of claim 325, wherein the Factor VIII analog lacks at least the portion of human Factor VIII B-domain corresponding to the region defined by (a) S750 to C1636 of human Factor VIII or (b) T760 to N1639 of human Factor VIII.

328. The method of claim 320, wherein the Factor VIII analog lacks one or more parts or all of the B-domain of human Factor VIII.

329. The method of claim 328, wherein the Factor VIII analog lacks from about 75% to about 85% or from about 85% to about 95% of the B-domain of human Factor VIII.

330. The method of claim 328, wherein the Factor VIII analog lacks at least the portion of human Factor VIII B-domain corresponding to the region defined by (a) S750 to C1636 or (b) T760 to N1639 of human Factor VIII.

331. The method of claim 315, wherein the Factor VIII analog is delivered to the patient by administering a pharmaceutical formulation comprising the Factor VIII analog and a pharmaceutically acceptable carrier to the patient.

332. The method of claim 317, wherein the Factor VIII analog is delivered to the patient by administering a pharmaceutical formulation comprising the Factor VIII analog and a pharmaceutically acceptable carrier to the patient.

333. The method of claim 332, wherein the method comprises once a week administration of the pharmaceutical formulation.

334. The method of claim 319, wherein the Factor VIII analog is delivered to the patient by administering a pharmaceutical formulation comprising the Factor VIII analog and a pharmaceutically acceptable carrier to the patient.

335. The method of claim 334, wherein the method comprises once a week administration of the pharmaceutical formulation.

336. The method of claim 320, wherein the Factor VIII analog is delivered to the patient by administering a pharmaceutical formulation comprising the Factor VIII analog and a pharmaceutically acceptable carrier to the patient.

337. The method of claim 322, wherein the Factor VIII analog is delivered to the patient by administering a pharmaceutical formulation comprising the Factor VIII analog and a pharmaceutically acceptable carrier to the patient.

338. The method of claim 326, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

339. The method of claim 338, wherein the water-soluble polymer is a PEG having an average molecular weight of 2-40 kDa and the method comprises once a week administration of the pharmaceutical formulation.

340. A Factor VIII analog comprising amino acid substitution(s) of at least one of the amino acid residues corresponding to the amino acid residues in positions 528-554 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

341. The Factor VIII analog of claim 340, wherein at least one of the amino acid residues corresponding to (i) V537, N538, A544, G546, or I548 and/or (ii) R541 of human Factor VIII is substituted with a Cys residue.

342. The Factor VIII analog of claim 341, wherein at least one of the substituting Cys residue(s) is conjugated to a water soluble polymer.

343. The Factor VIII analog of claim 342, wherein the water soluble polymer comprises a PEG.

344. The Factor VIII analog of claim 343, wherein the PEG has an average molecular weight of 2-40 kDa.

345. The Factor VIII analog of claim 340, wherein at least one N-glycosylation site is introduced into the Factor VIII analog (as compared to human Factor VIII) at a position starting at a residue that corresponds to a residue situation in a position of human Factor VIII selected from (i) V537, N538, A544, or G546; and/or (ii) R541 of the human FVIII molecule.

346. The Factor VIII analog of claim 341, wherein at least one N-glycosylation site is introduced into the Factor VIII analog (as compared to human Factor VIII) at a position starting at a residue that corresponds to a residue situation in a position of human Factor VIII selected from (i) V537, N538, A544, or G546; and/or (ii) R541 of the human FVIII molecule.

347. The Factor VIII analog of claim 340, wherein the Factor VIII analog lacks one or more parts or all of the B-domain of human Factor VIII.

348. The Factor VIII analog of claim 347, wherein the Factor VIII analog lacks from about 75% to about 85% or from about 85% to about 95% of the B-domain of human Factor VIII.

349. The Factor VIII of claim 347, wherein the Factor VIII analog lacks at least the portion of human Factor VIII B-domain corresponding to the region defined by (a) S750 to C1636 of human Factor VIII or (b) T760 to N1639 of human Factor VIII.

350. The Factor VIII analog of claim 341, wherein the Factor VIII analog lacks one or more parts or all of the B-domain of human Factor VIII.

351. The Factor VIII analog of claim 350, wherein the Factor VIII analog lacks from about 75% to about 85% or from about 85% to about 95% of the B-domain of human Factor VIII.

352. The Factor VIII of claim 350, wherein the Factor VIII analog lacks at least the portion of human Factor VIII B-domain corresponding to the region defined by (a) S750 to C1636 of human Factor VIII or (b) T760 to N1639 of human Factor VIII.

353. The Factor VIII analog of claim 345, wherein the Factor VIII analog lacks one or more parts or all of the B-domain of human Factor VIII.

354. The Factor VIII analog of claim 353, wherein the Factor VIII analog lacks from about 75% to about 85% or from about 85% to about 95% of the B-domain of human Factor VIII.

355. The Factor VIII of claim 353, wherein the Factor VIII analog lacks at least the portion of human Factor VIII B-domain corresponding to the region defined by (a) S750 to C1636 or (b) T760 to N1639 of human Factor VIII.

356. A pharmaceutical composition comprising a therapeutically effective amount of a Factor VIII analog according to claim 340 and a pharmaceutically acceptable carrier.

357. A pharmaceutical composition comprising a therapeutically effective amount of a Factor VIII analog according to claim 341 and a pharmaceutically acceptable carrier.

358. A pharmaceutical composition comprising a therapeutically effective amount of a Factor VIII analog according to claim 342 and a pharmaceutically acceptable carrier.

359. A pharmaceutical composition comprising a therapeutically effective amount of a Factor VIII analog according to claim 344 and a pharmaceutically acceptable carrier.

360. A pharmaceutical composition comprising a therapeutically effective amount of a Factor VIII analog according to claim 345 and a pharmaceutically acceptable carrier.

361. A pharmaceutical composition comprising a therapeutically effective amount of a Factor VIII analog according to claim 347 and a pharmaceutically acceptable carrier.

362. A pharmaceutical composition comprising a therapeutically effective amount of a Factor VIII analog according to claim 348 and a pharmaceutically acceptable carrier.

363. A pharmaceutical composition comprising a therapeutically effective amount of a Factor VIII analog according to claim 351 and a pharmaceutically acceptable carrier.

364. A pharmaceutical composition comprising (a) a therapeutically effective amount of a Factor VIII analog according to claim 351, wherein the substituting Cys residue is conjugated to a PEG having an average molecular weight of 2-40 kDa, and (b) a pharmaceutically acceptable carrier.

365. A method of treating a hemophilia patient comprising delivering to the patient a therapeutically effective amount of a Factor VIII analog comprising amino acid substitution(s) of at least one of the amino acid residues corresponding to the amino acid residues in positions 528-554 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

366. The method of claim 365, wherein at least one of the amino acid residues corresponding to (i) V537, N538, A544, or G546; and/or (ii) R541 of human Factor VIII is substituted with a Cys residue in the Factor VIII analog.

367. The method of claim 366, wherein at least one of the substituting Cys residue(s) is conjugated to a water soluble polymer.

368. The method of claim 367, wherein the water soluble polymer comprises a PEG.

369. The method of claim 368, wherein the PEG has an average molecular weight of 2-40 kDa.

370. The method of claim 365, wherein at least one N-glycosylation site is introduced into the Factor VIII analog (as compared to human Factor VIII) at a position starting at a residue that corresponds to a residue situation in a position of human Factor VIII selected from (i) V537, N538, A544, or G546; and/or (ii) R541 of the human FVIII molecule.

371. The method of claim 366, wherein at least one N-glycosylation site is introduced into the Factor VIII analog (as compared to human Factor VIII) at a position starting at a residue that corresponds to a residue situation in a position of human Factor VIII selected from (i) V537, N538, A544, or G546; and/or (ii) R541 of the human FVIII molecule.

372. The method of claim 365, wherein the Factor VIII analog lacks one or more parts or all of the B-domain of human Factor VIII.

373. The method of claim 372, wherein the Factor VIII analog lacks from about 75% to about 85% or from about 85% to about 95% of the B-domain of human Factor VIII.

374. The method of claim 372, wherein the Factor VIII analog lacks at least the portion of human Factor VIII B-domain corresponding to the region defined by (a) S750 to C1636 of human Factor VIII or (b) T760 to N1639 of human Factor VIII.

375. The method of claim 366, wherein the Factor VIII analog lacks one or more parts or all of the B-domain of human Factor VIII.

376. The method of claim 375, wherein the Factor VIII analog lacks from about 75% to about 85% or from about 85% to about 95% of the B-domain of human Factor VIII.

377. The method of claim 375, wherein the Factor VIII analog lacks at least the portion of human Factor VIII B-domain corresponding to the region defined by (a) S750 to C1636 of human Factor VIII or (b) T760 to N1639 of human Factor VIII.

378. The method of claim 370, wherein the Factor VIII analog lacks one or more parts or all of the B-domain of human Factor VIII.

379. The method of claim 378, wherein the Factor VIII analog lacks from about 75% to about 85% or from about 85% to about 95% of the B-domain of human Factor VIII.

380. The method of claim 378, wherein the Factor VIII analog lacks at least the portion of human Factor VIII B-domain corresponding to the region defined by (a) S750 to C1636 or (b) T760 to N1639 of human Factor VIII.

381. The method of claim 365, wherein the Factor VIII analog is delivered to the patient by administering a pharmaceutical formulation comprising the Factor VIII analog and a pharmaceutically acceptable carrier to the patient.

382. The method of claim 367, wherein the Factor VIII analog is delivered to the patient by administering a pharmaceutical formulation comprising the Factor VIII analog and a pharmaceutically acceptable carrier to the patient.

383. The method of claim 382, wherein the method comprises once a week administration of the pharmaceutical formulation.

384. The method of claim 369, wherein the Factor VIII analog is delivered to the patient by administering a pharmaceutical formulation comprising the Factor VIII analog and a pharmaceutically acceptable carrier to the patient.

385. The method of claim 384, wherein the method comprises once a week administration of the pharmaceutical formulation.

386. The method of claim 370, wherein the Factor VIII analog is delivered to the patient by administering a pharmaceutical formulation comprising the Factor VIII analog and a pharmaceutically acceptable carrier to the patient.

387. The method of claim 372, wherein the Factor VIII analog is delivered to the patient by administering a pharmaceutical formulation comprising the Factor VIII analog and a pharmaceutically acceptable carrier to the patient.

388. The method of claim 376, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

389. The method of claim 388, wherein the water-soluble polymer is a PEG having an average molecular weight of 2-40 kDa and the method comprises once a week administration of the pharmaceutical formulation.

390. A Factor VIII analog comprising amino acid substitution(s) of at least one of the amino acid residues corresponding to the amino acid residues in positions 559-564 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

391. The Factor VIII analog of claim 390, wherein at least one of the amino acid residues corresponding to (i) N564 and/or (ii) D560, Q561, or R562 of human Factor VIII is substituted with a Cys residue.

392. The Factor VIII analog of claim 391, wherein at least one of the substituting Cys residue(s) is conjugated to a water soluble polymer.

393. The Factor VIII analog of claim 392, wherein the water soluble polymer comprises a PEG.

394. The Factor VIII analog of claim 393, wherein the PEG has an average molecular weight of 2-40 kDa.

395. The Factor VIII analog of claim 390, wherein at least one N-glycosylation site is introduced into the Factor VIII analog (as compared to human Factor VIII) at a position starting at a residue that corresponds to a residue situation in a position of human Factor VIII selected from (i) N564 and/or (ii) D560, Q561, or R562 of the human FVIII molecule.

396. The Factor VIII analog of claim 391, wherein at least one N-glycosylation site is introduced into the Factor VIII analog (as compared to human Factor VIII) at a position starting at a residue that corresponds to a residue situation in a position of human Factor VIII selected from (i) N564 and/or (ii) D560, Q561, or R562 of the human FVIII molecule.

397. The Factor VIII analog of claim 390, wherein the Factor VIII analog lacks one or more parts or all of the B-domain of human Factor VIII.

398. The Factor VIII analog of claim 397, wherein the Factor VIII analog lacks from about 75% to about 85% or from about 85% to about 95% of the B-domain of human Factor VIII.

399. The Factor VIII of claim 397, wherein the Factor VIII analog lacks at least the portion of human Factor VIII B-domain corresponding to the region defined by (a) S750 to C1636 of human Factor VIII or (b) T760 to N1639 of human Factor VIII.

400. The Factor VIII analog of claim 391, wherein the Factor VIII analog lacks one or more parts or all of the B-domain of human Factor VIII.

401. The Factor VIII analog of claim 400, wherein the Factor VIII analog lacks from about 75% to about 85% or from about 85% to about 95% of the B-domain of human Factor VIII.

402. The Factor VIII of claim 400, wherein the Factor VIII analog lacks at least the portion of human Factor VIII B-domain corresponding to the region defined by (a) S750 to C1636 of human Factor VIII or (b) T760 to N1639 of human Factor VIII.

403. The Factor VIII analog of claim 395, wherein the Factor VIII analog lacks one or more parts or all of the B-domain of human Factor VIII.

404. The Factor VIII analog of claim 403, wherein the Factor VIII analog lacks from about 75% to about 85% or from about 85% to about 95% of the B-domain of human Factor VIII.

405. The Factor VIII of claim 403, wherein the Factor VIII analog lacks at least the portion of human Factor VIII B-domain corresponding to the region defined by (a) S750 to C1636 or (b) T760 to N1639 of human Factor VIII.

406. A pharmaceutical composition comprising a therapeutically effective amount of a Factor VIII analog according to claim 390 and a pharmaceutically acceptable carrier.

407. A pharmaceutical composition comprising a therapeutically effective amount of a Factor VIII analog according to claim 391 and a pharmaceutically acceptable carrier.

408. A pharmaceutical composition comprising a therapeutically effective amount of a Factor VIII analog according to claim 392 and a pharmaceutically acceptable carrier.

409. A pharmaceutical composition comprising a therapeutically effective amount of a Factor VIII analog according to claim 394 and a pharmaceutically acceptable carrier.

410. A pharmaceutical composition comprising a therapeutically effective amount of a Factor VIII analog according to claim 395 and a pharmaceutically acceptable carrier.

411. A pharmaceutical composition comprising a therapeutically effective amount of a Factor VIII analog according to claim 397 and a pharmaceutically acceptable carrier.

412. A pharmaceutical composition comprising a therapeutically effective amount of a Factor VIII analog according to claim 398 and a pharmaceutically acceptable carrier.

413. A pharmaceutical composition comprising a therapeutically effective amount of a Factor VIII analog according to claim 399 and a pharmaceutically acceptable carrier.

414. A pharmaceutical composition comprising (a) a therapeutically effective amount of a Factor VIII analog according to claim 401, wherein the substituting Cys residue is conjugated to a PEG having an average molecular weight of 2-40 kDa, and (b) a pharmaceutically acceptable carrier.

415. A method of treating a hemophilia patient comprising delivering to the patient a therapeutically effective amount of a Factor VIII analog comprising amino acid substitution(s) of at least one of the amino acid residues corresponding to the amino acid residues in positions 528-554 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

416. The method of claim 415, wherein at least one of the amino acid residues corresponding to (i) N564 and/or (ii) D560, Q561, or R562 of human Factor VIII is substituted with a Cys residue in the Factor VIII analog.

417. The method of claim 416, wherein at least one of the substituting Cys residue(s) is conjugated to a water soluble polymer.

418. The method of claim 417, wherein the water soluble polymer comprises a PEG.

419. The method of claim 418, wherein the PEG has an average molecular weight of 2-40 kDa.

420. The method of claim 415, wherein at least one N-glycosylation site is introduced into the Factor VIII analog (as compared to human Factor VIII) at a position starting at a residue that corresponds to a residue situation in a position of human Factor VIII selected from (i) N564 and/or (ii) D560, Q561, or R562 of the human FVIII molecule.

421. The method of claim 416, wherein at least one N-glycosylation site is introduced into the Factor VIII analog (as compared to human Factor VIII) at a position starting at a residue that corresponds to a residue situation in a position of human Factor VIII selected from (i) N564 and/or (ii) D560, Q561, or R562 of the human FVIII molecule.

422. The method of claim 415, wherein the Factor VIII analog lacks one or more parts or all of the B-domain of human Factor VIII.

423. The method of claim 422, wherein the Factor VIII analog lacks from about 75% to about 85% or from about 85% to about 95% of the B-domain of human Factor VIII.

424. The method of claim 422, wherein the Factor VIII analog lacks at least the portion of human Factor VIII B-domain corresponding to the region defined by (a) S750 to C1636 of human Factor VIII or (b) T760 to N1639 of human Factor VIII.

425. The method of claim 416, wherein the Factor VIII analog lacks one or more parts or all of the B-domain of human Factor VIII.

426. The method of claim 425, wherein the Factor VIII analog lacks from about 75% to about 85% or from about 85% to about 95% of the B-domain of human Factor VIII.

427. The method of claim 425, wherein the Factor VIII analog lacks at least the portion of human Factor VIII B-domain corresponding to the region defined by (a) S750 to C1636 of human Factor VIII or (b) T760 to N1639 of human Factor VIII.

428. The method of claim 420, wherein the Factor VIII analog lacks one or more parts or all of the B-domain of human Factor VIII.

429. The method of claim 428, wherein the Factor VIII analog lacks from about 75% to about 85% or from about 85% to about 95% of the B-domain of human Factor VIII.

430. The method of claim 428, wherein the Factor VIII analog lacks at least the portion of human Factor VIII B-domain corresponding to the region defined by (a) S750 to C1636 or (b) T760 to N1639 of human Factor VIII.

431. The method of claim 415, wherein the Factor VIII analog is delivered to the patient by administering a pharmaceutical formulation comprising the Factor VIII analog and a pharmaceutically acceptable carrier to the patient.

432. The method of claim 417, wherein the Factor VIII analog is delivered to the patient by administering a pharmaceutical formulation comprising the Factor VIII analog and a pharmaceutically acceptable carrier to the patient.

433. The method of claim 432, wherein the method comprises once a week administration of the pharmaceutical formulation.

434. The method of claim 419, wherein the Factor VIII analog is delivered to the patient by administering a pharmaceutical formulation comprising the Factor VIII analog and a pharmaceutically acceptable carrier to the patient.

435. The method of claim 434, wherein the method comprises once a week administration of the pharmaceutical formulation.

436. The method of claim 420, wherein the Factor VIII analog is delivered to the patient by administering a pharmaceutical formulation comprising the Factor VIII analog and a pharmaceutically acceptable carrier to the patient.

437. The method of claim 422, wherein the Factor VIII analog is delivered to the patient by administering a pharmaceutical formulation comprising the Factor VIII analog and a pharmaceutically acceptable carrier to the patient.

438. The method of claim 426, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

439. The method of claim 438, wherein the water-soluble polymer is a PEG having an average molecular weight of 2-40 kDa and the method comprises once a week administration of the pharmaceutical formulation.

440. A Factor VIII analog comprising amino acid substitution(s) of at least one of the amino acid residues corresponding to the amino acid residues in positions 571-593 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

441. The Factor VIII analog of claim 440, wherein at least one of the amino acid residues corresponding to (i) N572, F576, V578, W585, or L587; and/or (ii) D580, R583, or E589; and/or (iii) T588, Q592, or R593 of human Factor VIII is substituted with a Cys residue.

442. The Factor VIII analog of claim 441, wherein at least one of the substituting Cys residue(s) is conjugated to a water soluble polymer.

443. The Factor VIII analog of claim 442, wherein the water soluble polymer comprises a PEG.

444. The Factor VIII analog of claim 443, wherein the PEG has an average molecular weight of 2-40 kDa.

445. The Factor VIII analog of claim 440, wherein at least one N-glycosylation site is introduced into the Factor VIII analog (as compared to human Factor VIII) at a position starting at a residue that corresponds to a residue situation in a position of human Factor VIII selected from (i) N572, F576, V578, W585, or L587; and/or (ii) D580, R583, or E589; and/or (iii) T588, Q592, or R593 of the human FVIII molecule.

446. The Factor VIII analog of claim 441, wherein at least one N-glycosylation site is introduced into the Factor VIII analog (as compared to human Factor VIII) at a position starting at a residue that corresponds to a residue situation in a position of human Factor VIII selected from (i) N572, F576, V578, W585, or L587; and/or (ii) D580, R583, or E589; and/or (iii) T588, Q592, or R593 of the human FVIII molecule.

447. The Factor VIII analog of claim 440, wherein the Factor VIII analog lacks one or more parts or all of the B-domain of human Factor VIII.

448. The Factor VIII analog of claim 447, wherein the Factor VIII analog lacks from about 75% to about 85% or from about 85% to about 95% of the B-domain of human Factor VIII.

449. The Factor VIII of claim 447, wherein the Factor VIII analog lacks at least the portion of human Factor VIII B-domain corresponding to the region defined by (a) S750 to C1636 of human Factor VIII or (b) T760 to N1639 of human Factor VIII.

450. The Factor VIII analog of claim 441, wherein the Factor VIII analog lacks one or more parts or all of the B-domain of human Factor VIII.

451. The Factor VIII analog of claim 450, wherein the Factor VIII analog lacks from about 75% to about 85% or from about 85% to about 95% of the B-domain of human Factor VIII.

452. The Factor VIII of claim 450, wherein the Factor VIII analog lacks at least the portion of human Factor VIII B-domain corresponding to the region defined by (a) S750 to C1636 of human Factor VIII or (b) T760 to N1639 of human Factor VIII.

453. The Factor VIII analog of claim 445, wherein the Factor VIII analog lacks one or more parts or all of the B-domain of human Factor VIII.

454. The Factor VIII analog of claim 453, wherein the Factor VIII analog lacks from about 75% to about 85% or from about 85% to about 95% of the B-domain of human Factor VIII.

455. The Factor VIII of claim 453, wherein the Factor VIII analog lacks at least the portion of human Factor VIII B-domain corresponding to the region defined by (a) S750 to C1636 or (b) T760 to N1639 of human Factor VIII.

456. A pharmaceutical composition comprising a therapeutically effective amount of a Factor VIII analog according to claim 440 and a pharmaceutically acceptable carrier.

457. A pharmaceutical composition comprising a therapeutically effective amount of a Factor VIII analog according to claim 441 and a pharmaceutically acceptable carrier.

458. A pharmaceutical composition comprising a therapeutically effective amount of a Factor VIII analog according to claim 442 and a pharmaceutically acceptable carrier.

459. A pharmaceutical composition comprising a therapeutically effective amount of a Factor VIII analog according to claim 444 and a pharmaceutically acceptable carrier.

460. A pharmaceutical composition comprising a therapeutically effective amount of a Factor VIII analog according to claim 445 and a pharmaceutically acceptable carrier.

461. A pharmaceutical composition comprising a therapeutically effective amount of a Factor VIII analog according to claim 447 and a pharmaceutically acceptable carrier.

462. A pharmaceutical composition comprising a therapeutically effective amount of a Factor VIII analog according to claim 448 and a pharmaceutically acceptable carrier.

463. A pharmaceutical composition comprising a therapeutically effective amount of a Factor VIII analog according to claim 449 and a pharmaceutically acceptable carrier.

464. A pharmaceutical composition comprising (a) a therapeutically effective amount of a Factor VIII analog according to claim 451, wherein the substituting Cys residue is conjugated to a PEG having an average molecular weight of 2-40 kDa, and (b) a pharmaceutically acceptable carrier.

465. A method of treating a hemophilia patient comprising delivering to the patient a therapeutically effective amount of a Factor VIII analog comprising amino acid substitution(s) of at least one of the amino acid residues corresponding to the amino acid residues in positions 571-593 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

466. The method of claim 465, wherein at least one of the amino acid residues corresponding to (i) N572, F576, V578, W585, or L587; and/or (ii) D580, R583, or E589; and/or (iii) T588, Q592, or R593 of human Factor VIII is substituted with a Cys residue in the Factor VIII analog.

467. The method of claim 466, wherein at least one of the substituting Cys residue(s) is conjugated to a water soluble polymer.

468. The method of claim 467, wherein the water soluble polymer comprises a PEG.

469. The method of claim 468, wherein the PEG has an average molecular weight of 2-40 kDa.

470. The method of claim 465, wherein at least one N-glycosylation site is introduced into the Factor VIII analog (as compared to human Factor VIII) at a position starting at a residue that corresponds to a residue situation in a position of human Factor VIII selected from (i) N572, F576, V578, W585, or L587; and/or (ii) D580, R583, or E589; and/or (iii) T588, Q592, or R593 of the human FVIII molecule.

471. The method of claim 466, wherein at least one N-glycosylation site is introduced into the Factor VIII analog (as compared to human Factor VIII) at a position starting at a residue that corresponds to a residue situation in a position of human Factor VIII selected from (i) N572, F576, V578, W585, or L587; and/or (ii) D580, R583, or E589; and/or (iii) T588, Q592, or R593 of the human FVIII molecule.

472. The method of claim 465, wherein the Factor VIII analog lacks one or more parts or all of the B-domain of human Factor VIII.

473. The method of claim 472, wherein the Factor VIII analog lacks from about 75% to about 85% or from about 85% to about 95% of the B-domain of human Factor VIII.

474. The method of claim 472, wherein the Factor VIII analog lacks at least the portion of human Factor VIII B-domain corresponding to the region defined by (a) S750 to C1636 of human Factor VIII or (b) T760 to N1639 of human Factor VIII.

475. The method of claim 466, wherein the Factor VIII analog lacks one or more parts or all of the B-domain of human Factor VIII.

476. The method of claim 475, wherein the Factor VIII analog lacks from about 75% to about 85% or from about 85% to about 95% of the B-domain of human Factor VIII.

477. The method of claim 475, wherein the Factor VIII analog lacks at least the portion of human Factor VIII B-domain corresponding to the region defined by (a) S750 to C1636 of human Factor VIII or (b) T760 to N1639 of human Factor VIII.

478. The method of claim 470, wherein the Factor VIII analog lacks one or more parts or all of the B-domain of human Factor VIII.

479. The method of claim 478, wherein the Factor VIII analog lacks from about 75% to about 85% or from about 85% to about 95% of the B-domain of human Factor VIII.

480. The method of claim 478, wherein the Factor VIII analog lacks at least the portion of human Factor VIII B-domain corresponding to the region defined by (a) S750 to C1636 or (b) T760 to N1639 of human Factor VIII.

481. The method of claim 465, wherein the Factor VIII analog is delivered to the patient by administering a pharmaceutical formulation comprising the Factor VIII analog and a pharmaceutically acceptable carrier to the patient.

482. The method of claim 467, wherein the Factor VIII analog is delivered to the patient by administering a pharmaceutical formulation comprising the Factor VIII analog and a pharmaceutically acceptable carrier to the patient.

483. The method of claim 482, wherein the method comprises once a week administration of the pharmaceutical formulation.

484. The method of claim 469, wherein the Factor VIII analog is delivered to the patient by administering a pharmaceutical formulation comprising the Factor VIII analog and a pharmaceutically acceptable carrier to the patient.

485. The method of claim 484, wherein the method comprises once a week administration of the pharmaceutical formulation.

486. The method of claim 470, wherein the Factor VIII analog is delivered to the patient by administering a pharmaceutical formulation comprising the Factor VIII analog and a pharmaceutically acceptable carrier to the patient.

487. The method of claim 472, wherein the Factor VIII analog is delivered to the patient by administering a pharmaceutical formulation comprising the Factor VIII analog and a pharmaceutically acceptable carrier to the patient.

488. The method of claim 476, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

489. The method of claim 488, wherein the water-soluble polymer is a PEG having an average molecular weight of 2-40 kDa and the method comprises once a week administration of the pharmaceutical formulation.

490. A Factor VIII analog comprising amino acid substitution(s) of at least one of the amino acid residues corresponding to the amino acid residues in positions 638-643 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

491. The Factor VIII analog of claim 490, wherein at least one of the amino acid residues corresponding to 1639, S641, and/or G643 of human Factor VIII is substituted with a Cys residue.

492. The Factor VIII analog of claim 491, wherein at least one of the substituting Cys residue(s) is conjugated to a water soluble polymer.

493. The Factor VIII analog of claim 492, wherein the water soluble polymer comprises a PEG.

494. The Factor VIII analog of claim 493, wherein the PEG has an average molecular weight of 2-40 kDa.

495. The Factor VIII analog of claim 490, wherein at least one N-glycosylation site is introduced into the Factor VIII analog (as compared to human Factor VIII) at a position starting at a residue that corresponds to a residue situation in a position of human Factor VIII selected from 1639, S641, and G643 of the human FVIII molecule.

496. The Factor VIII analog of claim 491, wherein at least one N-glycosylation site is introduced into the Factor VIII analog (as compared to human Factor VIII) at a position starting at a residue that corresponds to a residue situation in a position of human Factor VIII selected from 1639, S641, and G643 of the human FVIII molecule.

497. The Factor VIII analog of claim 490, wherein the Factor VIII analog lacks one or more parts or all of the B-domain of human Factor VIII.

498. The Factor VIII analog of claim 497, wherein the Factor VIII analog lacks from about 75% to about 85% or from about 85% to about 95% of the B-domain of human Factor VIII.

499. The Factor VIII of claim 497, wherein the Factor VIII analog lacks at least the portion of human Factor VIII B-domain corresponding to the region defined by (a) S750 to C1636 of human Factor VIII or (b) T760 to N1639 of human Factor VIII.

500. The Factor VIII analog of claim 491, wherein the Factor VIII analog lacks one or more parts or all of the B-domain of human Factor VIII.

501. The Factor VIII analog of claim 500, wherein the Factor VIII analog lacks from about 75% to about 85% or from about 85% to about 95% of the B-domain of human Factor VIII.

502. The Factor VIII of claim 500, wherein the Factor VIII analog lacks at least the portion of human Factor VIII B-domain corresponding to the region defined by (a) S750 to C1636 of human Factor VIII or (b) T760 to N1639 of human Factor VIII.

503. The Factor VIII analog of claim 495, wherein the Factor VIII analog lacks one or more parts or all of the B-domain of human Factor VIII.

504. The Factor VIII analog of claim 503, wherein the Factor VIII analog lacks from about 75% to about 85% or from about 85% to about 95% of the B-domain of human Factor VIII.

505. The Factor VIII of claim 503, wherein the Factor VIII analog lacks at least the portion of human Factor VIII B-domain corresponding to the region defined by (a) S750 to C1636 or (b) T760 to N1639 of human Factor VIII.

506. A pharmaceutical composition comprising a therapeutically effective amount of a Factor VIII analog according to claim 490 and a pharmaceutically acceptable carrier.

507. A pharmaceutical composition comprising a therapeutically effective amount of a Factor VIII analog according to claim 491 and a pharmaceutically acceptable carrier.

508. A pharmaceutical composition comprising a therapeutically effective amount of a Factor VIII analog according to claim 492 and a pharmaceutically acceptable carrier.

509. A pharmaceutical composition comprising a therapeutically effective amount of a Factor VIII analog according to claim 494 and a pharmaceutically acceptable carrier.

510. A pharmaceutical composition comprising a therapeutically effective amount of a Factor VIII analog according to claim 495 and a pharmaceutically acceptable carrier.

511. A pharmaceutical composition comprising a therapeutically effective amount of a Factor VIII analog according to claim 497 and a pharmaceutically acceptable carrier.

512. A pharmaceutical composition comprising a therapeutically effective amount of a Factor VIII analog according to claim 498 and a pharmaceutically acceptable carrier.

513. A pharmaceutical composition comprising a therapeutically effective amount of a Factor VIII analog according to claim 499 and a pharmaceutically acceptable carrier.

514. A pharmaceutical composition comprising (a) a therapeutically effective amount of a Factor VIII analog according to claim 501, wherein the substituting Cys residue is conjugated to a PEG having an average molecular weight of 2-40 kDa, and (b) a pharmaceutically acceptable carrier.

515. A method of treating a hemophilia patient comprising delivering to the patient a therapeutically effective amount of a Factor VIII analog comprising amino acid substitution(s) of at least one of the amino acid residues corresponding to the amino acid residues in positions 638-643 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

516. The method of claim 515, wherein at least one of the amino acid residues corresponding to 1639, S641, and/or G643 of human Factor VIII is substituted with a Cys residue in the Factor VIII analog.

517. The method of claim 516, wherein at least one of the substituting Cys residue(s) is conjugated to a water soluble polymer.

518. The method of claim 517, wherein the water soluble polymer comprises a PEG.

519. The method of claim 518, wherein the PEG has an average molecular weight of 2-40 kDa.

520. The method of claim 515, wherein at least one N-glycosylation site is introduced into the Factor VIII analog (as compared to human Factor VIII) at a position starting at a residue that corresponds to a residue situation in a position of human Factor VIII selected from 1639, S641, and G643 of the human FVIII molecule.

521. The method of claim 516, wherein at least one N-glycosylation site is introduced into the Factor VIII analog (as compared to human Factor VIII) at a position starting at a residue that corresponds to a residue situation in a position of human Factor VIII selected from 1639, S641, and G643 of the human FVIII molecule.

522. The method of claim 515, wherein the Factor VIII analog lacks one or more parts or all of the B-domain of human Factor VIII.

523. The method of claim 522, wherein the Factor VIII analog lacks from about 75% to about 85% or from about 85% to about 95% of the B-domain of human Factor VIII.

524. The method of claim 522, wherein the Factor VIII analog lacks at least the portion of human Factor VIII B-domain corresponding to the region defined by (a) S750 to C1636 of human Factor VIII or (b) T760 to N1639 of human Factor VIII.

525. The method of claim 516, wherein the Factor VIII analog lacks one or more parts or all of the B-domain of human Factor VIII.

526. The method of claim 525, wherein the Factor VIII analog lacks from about 75% to about 85% or from about 85% to about 95% of the B-domain of human Factor VIII.

527. The method of claim 525, wherein the Factor VIII analog lacks at least the portion of human Factor VIII B-domain corresponding to the region defined by (a) S750 to C1636 of human Factor VIII or (b) T760 to N1639 of human Factor VIII.

528. The method of claim 520, wherein the Factor VIII analog lacks one or more parts or all of the B-domain of human Factor VIII.

529. The method of claim 528, wherein the Factor VIII analog lacks from about 75% to about 85% or from about 85% to about 95% of the B-domain of human Factor VIII.

530. The method of claim 528, wherein the Factor VIII analog lacks at least the portion of human Factor VIII B-domain corresponding to the region defined by (a) S750 to C1636 or (b) T760 to N1639 of human Factor VIII.

531. The method of claim 515, wherein the Factor VIII analog is delivered to the patient by administering a pharmaceutical formulation comprising the Factor VIII analog and a pharmaceutically acceptable carrier to the patient.

532. The method of claim 517, wherein the Factor VIII analog is delivered to the patient by administering a pharmaceutical formulation comprising the Factor VIII analog and a pharmaceutically acceptable carrier to the patient.

533. The method of claim 532, wherein the method comprises once a week administration of the pharmaceutical formulation.

534. The method of claim 519, wherein the Factor VIII analog is delivered to the patient by administering a pharmaceutical formulation comprising the Factor VIII analog and a pharmaceutically acceptable carrier to the patient.

535. The method of claim 534, wherein the method comprises once a week administration of the pharmaceutical formulation.

536. The method of claim 520, wherein the Factor VIII analog is delivered to the patient by administering a pharmaceutical formulation comprising the Factor VIII analog and a pharmaceutically acceptable carrier to the patient.

537. The method of claim 522, wherein the Factor VIII analog is delivered to the patient by administering a pharmaceutical formulation comprising the Factor VIII analog and a pharmaceutically acceptable carrier to the patient.

538. The method of claim 526, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

539. The method of claim 538, wherein the water-soluble polymer is a PEG having an average molecular weight of 2-40 kDa and the method comprises once a week administration of the pharmaceutical formulation.

540. A Factor VIII analog comprising amino acid substitution(s) of at least one of the amino acid residues corresponding to the amino acid residues in positions K422, R427, M429, and/or S674 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

541. The Factor VIII analog of claim 540, wherein at least one of the substitution(s) is a Cys residue substitution.

542. The Factor VIII analog of claim 541, wherein at least one of the substituting Cys residue(s) is conjugated to a water soluble polymer.

543. The Factor VIII analog of claim 542, wherein the water soluble polymer comprises a PEG.

544. The Factor VIII analog of claim 543, wherein the PEG has an average molecular weight of 2-40 kDa.

545. The Factor VIII analog of claim 540, wherein at least one N-glycosylation site is introduced into the Factor VIII analog (as compared to human Factor VIII) at a position starting at a residue that corresponds to a residue situation in a position of human Factor VIII selected from K422, R427, M429, and S674 of the human FVIII molecule.

546. The Factor VIII analog of claim 541, wherein at least one N-glycosylation site is introduced into the Factor VIII analog (as compared to human Factor VIII) at a position starting at a residue that corresponds to a residue situation in a position of human Factor VIII selected from K422, R427, M429, and S674 of the human FVIII molecule.

547. The Factor VIII analog of claim 540, wherein the Factor VIII analog lacks one or more parts or all of the B-domain of human Factor VIII.

548. The Factor VIII analog of claim 547, wherein the Factor VIII analog lacks from about 75% to about 85% or from about 85% to about 95% of the B-domain of human Factor VIII.

549. The Factor VIII of claim 547, wherein the Factor VIII analog lacks at least the portion of human Factor VIII B-domain corresponding to the region defined by (a) S750 to C1636 of human Factor VIII or (b) T760 to N1639 of human Factor VIII.

550. The Factor VIII analog of claim 541, wherein the Factor VIII analog lacks one or more parts or all of the B-domain of human Factor VIII.

551. The Factor VIII analog of claim 550, wherein the Factor VIII analog lacks from about 75% to about 85% or from about 85% to about 95% of the B-domain of human Factor VIII.

552. The Factor VIII of claim 550, wherein the Factor VIII analog lacks at least the portion of human Factor VIII B-domain corresponding to the region defined by (a) S750 to C1636 of human Factor VIII or (b) T760 to N1639 of human Factor VIII.

553. The Factor VIII analog of claim 545, wherein the Factor VIII analog lacks one or more parts or all of the B-domain of human Factor VIII.

554. The Factor VIII analog of claim 553, wherein the Factor VIII analog lacks from about 75% to about 85% or from about 85% to about 95% of the B-domain of human Factor VIII.

555. The Factor VIII of claim 553, wherein the Factor VIII analog lacks at least the portion of human Factor VIII B-domain corresponding to the region defined by (a) S750 to C1636 or (b) T760 to N1639 of human Factor VIII.

556. A pharmaceutical composition comprising a therapeutically effective amount of a Factor VIII analog according to claim 540 and a pharmaceutically acceptable carrier.

557. A pharmaceutical composition comprising a therapeutically effective amount of a Factor VIII analog according to claim 541 and a pharmaceutically acceptable carrier.

558. A pharmaceutical composition comprising a therapeutically effective amount of a Factor VIII analog according to claim 542 and a pharmaceutically acceptable carrier.

559. A pharmaceutical composition comprising a therapeutically effective amount of a Factor VIII analog according to claim 544 and a pharmaceutically acceptable carrier.

560. A pharmaceutical composition comprising a therapeutically effective amount of a Factor VIII analog according to claim 545 and a pharmaceutically acceptable carrier.

561. A pharmaceutical composition comprising a therapeutically effective amount of a Factor VIII analog according to claim 547 and a pharmaceutically acceptable carrier.

562. A pharmaceutical composition comprising a therapeutically effective amount of a Factor VIII analog according to claim 548 and a pharmaceutically acceptable carrier.

563. A pharmaceutical composition comprising a therapeutically effective amount of a Factor VIII analog according to claim 549 and a pharmaceutically acceptable carrier.

564. A pharmaceutical composition comprising (a) a therapeutically effective amount of a Factor VIII analog according to claim 551, wherein the substituting Cys residue is conjugated to a PEG having an average molecular weight of 2-40 kDa, and (b) a pharmaceutically acceptable carrier.

565. A method of treating a hemophilia patient comprising delivering to the patient a therapeutically effective amount of a Factor VIII analog comprising amino acid substitution(s) of at least one of the amino acid residues corresponding to K422, R427, M429, and/or S674 of the human Factor VIII molecule, which amino acid substitution(s) confer(s) the Factor VIII analog with an LRP binding affinity that is lower than that of human Factor VIII while not substantially reducing the Factor VIII activity of the Factor VIII analog as compared to human Factor VIII.

566. The method of claim 565, wherein at least one of the substitution(s) is a Cys residue substitution.

567. The method of claim 566, wherein at least one of the substituting Cys residue(s) is conjugated to a water soluble polymer.

568. The method of claim 567, wherein the water soluble polymer comprises a PEG.

569. The method of claim 568, wherein the PEG has an average molecular weight of 2-40 kDa.

570. The method of claim 565, wherein at least one N-glycosylation site is introduced into the Factor VIII analog (as compared to human Factor VIII) at a position starting at a residue that corresponds to a residue situation in a position of human Factor VIII selected from K422, R427, M429, and S674 of the human FVIII molecule.

571. The method of claim 566, wherein at least one N-glycosylation site is introduced into the Factor VIII analog (as compared to human Factor VIII) at a position starting at a residue that corresponds to a residue situation in a position of human Factor VIII selected from K422, R427, M429, and S674 of the human FVIII molecule.

572. The method of claim 565, wherein the Factor VIII analog lacks one or more parts or all of the B-domain of human Factor VIII.

573. The method of claim 572, wherein the Factor VIII analog lacks from about 75% to about 85% or from about 85% to about 95% of the B-domain of human Factor VIII.

574. The method of claim 572, wherein the Factor VIII analog lacks at least the portion of human Factor VIII B-domain corresponding to the region defined by (a) S750 to C1636 of human Factor VIII or (b) T760 to N1639 of human Factor VIII.

575. The method of claim 566, wherein the Factor VIII analog lacks one or more parts or all of the B-domain of human Factor VIII.

576. The method of claim 575, wherein the Factor VIII analog lacks from about 75% to about 85% or from about 85% to about 95% of the B-domain of human Factor VIII.

577. The method of claim 575, wherein the Factor VIII analog lacks at least the portion of human Factor VIII B-domain corresponding to the region defined by (a) S750 to C1636 of human Factor VIII or (b) T760 to N1639 of human Factor VIII.

578. The method of claim 570, wherein the Factor VIII analog lacks one or more parts or all of the B-domain of human Factor VIII.

579. The method of claim 578, wherein the Factor VIII analog lacks from about 75% to about 85% or from about 85% to about 95% of the B-domain of human Factor VIII.

580. The method of claim 578, wherein the Factor VIII analog lacks at least the portion of human Factor VIII B-domain corresponding to the region defined by (a) S750 to C1636 or (b) T760 to N1639 of human Factor VIII.

581. The method of claim 565, wherein the Factor VIII analog is delivered to the patient by administering a pharmaceutical formulation comprising the Factor VIII analog and a pharmaceutically acceptable carrier to the patient.

582. The method of claim 567, wherein the Factor VIII analog is delivered to the patient by administering a pharmaceutical formulation comprising the Factor VIII analog and a pharmaceutically acceptable carrier to the patient.

583. The method of claim 582, wherein the method comprises once a week administration of the pharmaceutical formulation.

584. The method of claim 569, wherein the Factor VIII analog is delivered to the patient by administering a pharmaceutical formulation comprising the Factor VIII analog and a pharmaceutically acceptable carrier to the patient.

585. The method of claim 584, wherein the method comprises once a week administration of the pharmaceutical formulation.

586. The method of claim 570, wherein the Factor VIII analog is delivered to the patient by administering a pharmaceutical formulation comprising the Factor VIII analog and a pharmaceutically acceptable carrier to the patient.

587. The method of claim 572, wherein the Factor VIII analog is delivered to the patient by administering a pharmaceutical formulation comprising the Factor VIII analog and a pharmaceutically acceptable carrier to the patient.

588. The method of claim 576, wherein at least one of the substituting Cys residue(s) is conjugated to a water soluble polymer in the Factor VIII analog and the Factor VIII analog is delivered to the patient by administering a pharmaceutical formulation comprising the Factor VIII analog and a pharmaceutically acceptable carrier to the patient.

589. The method of claim 588, wherein the water-soluble polymer is a PEG having an average molecular weight of 2-40 kDa and the method comprises once a week administration of the pharmaceutical formulation.
